(19) United States
(12) Patent Application Publication
(10) Pub. No.: US 2013/0078678 A1
(43) Pub. Date: Mar. 28, 2013

(54) Protozoan Glycosidases and Related Methods

(71) Applicants: Gary Stacey, Columbia, MO (US); Seth D. Findley, Coral Gables, FL (US); Melanie Mormile, Rolla, MO (US)

(72) Inventors: Gary Stacey, Columbia, MO (US); Seth D. Findley, Coral Gables, FL (US); Melanie Mormile, Rolla, MO (US)

(73) Assignee: THE CURATORS OF THE UNIVERSITY OF MISSOURI, Columbia, MO (US)

(21) Appl. No.: 13/619,376

(22) Filed: Sep. 14, 2012

Related U.S. Application Data
(60) Provisional application No. 61/573,892, filed on Sep. 14, 2011.

(51) Int. Cl. C12N 9/42

(52) U.S. Cl. CPC C12N 9/42
USPC 435/99; 435/320.1; 435/252.3; 435/254.2; 435/257.2; 435/258.1; 435/419; 435/254.11; 435/325; 435/209

(57) ABSTRACT

Nucleic acids encoding glycosidases useful for the hydrolysis of cellulose and hemicellulose obtained from ciliates residing in a bovine rumen are provided. Also provided recombinant nucleic acids encoding the glycosidases, transformed cells comprising the same and related methods of using the transformed cells and glycosidases to degrade cellulose and/or hemicellulose.

### CROSS-REFERENCE TO RELATED PATENT APPLICATIONS

This non-provisional US patent application claims the benefit of U.S. Provisional Patent Application No. 61/573,892, which was filed Sep. 14, 2011 and which is incorporated herein by reference in its entirety.

### SEQUENCE LISTING STATEMENT

The sequence listing that is contained in the file named “52553_{—}107399_ST25.txt”, which is 43,940 bytes (measured in operating system MS-Windows), created on Sep. 14, 2012, is filed herewith by electronic submission and incorporated herein by reference in its entirety. The sequence listing contains SEQ ID NO: 1-18.

### GRANT STATEMENT

None.

### FIELD OF INVENTION

The present invention relates to glycoside hydrolase enzymes. Recombinant nucleic acids containing a cDNA that encodes a novel and highly active arabinoxylanase or a cDNA that encodes a novel and highly active xyloglucanase are provided. Glycoside hydrolases provided herein find a variety of uses including the direct enzymatic processing of the lignocellulosic materials derived from plant feed stock. Recombinant gene cassettes for expression of the glycoside hydrolase enzymes by recombinant microbes that function to carry out such processes in bioreactors are also provided.

### BACKGROUND OF INVENTION

The rumen, the foregut of herbivorous ruminant animals, such as cattle, functions as a bioreactor to process complex plant material, and fibrolytic enzymes are essential for the digestion of cellulosic biomass in the ruminant diet. A suite of enzymes is required to produce a variety of free sugars, (exo-enzymes), as well as oligosaccharides (endo-enzymes) for metabolism by rumen bacteria and subsequent digestion by the ruminant host. Among the numerous and diverse microbes involved in ruminal digestion are the ruminal protozoans, which are single-celled, ciliated eukaryotic organisms.

Also, a broad range of specific classes of glycoside hydrolases are required to effect processing of biofuel feedstocks. One of the critical, time-consuming and rate-limiting steps in development of industrial-scale biofuel production is identification and biochemical characterization of the diverse glycosyl hydrolases required.

To process complex fibrous plant materials, the rumen harbors a complex collection of diverse microorganisms (reviewed by 33, 45). While the diversity and functions of the thousands (32) of microbial species of this unique ecosystem are interesting from both evolutionary and functional perspectives, the rumen also represents a rich resource of enzymes for converting lignocellulosic feedstocks into biofuel (35, 43) and other applications (19). A range of inexpensive, robust enzymes with a broad range of specificities will likely be required for efficient industrial processing of highly complex plant polysaccharides. Identification of such enzymes that microorganisms use to break down plant materials has been greatly facilitated by metagenomics (42), both in the form of activity-based screens (20, 52) or through increasingly powerful, high-throughput genomic DNA sequencing approaches (e.g., 28, 57). As evidenced by numerous studies (e.g., 28, 39, 41), metagenomics has proven to be particularly effective for identification of carbohydrate-active genes of fiber-adherent bacterial species of the rumen.

In addition to bacteria and archaea, the rumen also hosts eukaryotic species, namely anaerobic fungi and ciliate protozoa (reviewed by 33). Addressing the function of ruminal protozoa in particular has been a challenge due to the difficulty of maintaining these organisms in axenic cultures (55). Thus, assessing the diversity and dynamics of ruminal protozoa has been addressed historically by morphogenic studies (reviewed by 12) and molecular phylogenetics (e.g., using 18S rDNA markers; 47). Ruminal protozoa are known to contribute to fiber degradation in their hosts (21), and determination and characterization of their ability to directly process plant material has been addressed by diverse strategies, such as direct, biochemical detection of specific fibrolytic enzymes (e.g., cellulases) in extracts derived from individual protozoan species (e.g., 38, 54), by molecular cloning studies to directly identify genes encoding enzymes capable of degrading cellulose or hemicellulose (e.g., 49, 50) and, most recently, by sequencing of protozoan-derived EST libraries (41). Early studies to establish the capacity of protozoan species to express their own enzymes for degradation of plant material includes that of Howard et al. (29), who demonstrated that Epidinium ecaudatum (E. ecaudatum) indeed contains fibrolytic enzyme activity. Similarly, Bailey et al. (3), demonstrated the presence of both a hemicellulase and a xylobiase in E. ecaudatum using purified cell extracts. More recently, Clayet et al. (8), using gel filtration of E. ecaudatum extracts, identified at least ten distinct enzyme activities for plant cell wall degradation; their fractions contained a range of enzymes with glycoside hydrolase (GH) activities, including two distinct carboxymethylcellulases with molecular weights of 23 and 45 kDa (8).

Altogether, about a dozen protozoan fibrolytic genes have been identified in activity-based molecular screens; a comparable number have been identified in informatics-based studies (41), predominantly in ovine and bovine rumen systems. The protozoan enzyme genes characterized to date are diverse, both in terms of the individual GH domains (27) utilized, as well as the combinatorial domain organization of proteins that contain them. GH domains are modular by design; they exist in individual polypeptides in variable copy numbers and variable association with other, non-catalytic modules (e.g., carbohydrate binding domains; reviewed in 27). The described rumen protozoan-derived GH domain genes primarily encode single- or dual-GH-5 domains (cellulase superfamily; e.g., 49, 50, 53), or GH10 or GH11 domains (xylanase-related domains; e.g., 4, 14, 15). The combinatorial complexity of fibrolyic genes thus far detected in ruminal ciliates speaks to the potentially diverse utilization of GH modules within the entire ruminal protozoan population (4, 14, 15, 41, 49, 50, 53). Yet, due in part to the importance of demonstrating the existence of fibrolytic genes in a given protozoan species, enzyme cloning studies have largely been conducted using mono-faunated animals, in which the host ruminant is inoculated with a single ciliate species.

Therefore, there is a need to identify and provide cDNA encoding the enzyme with a substrate specificity that is valuable in the development of various industrial processes for the processing of lignocellulytic materials derived from plant feed stocks.

### SUMMARY

The invention provides a bovine protozoan glycoside hydrolase cDNA with a substrate specificity that is highly valuable in the development of various industrial processes for the processing of lignocellulytic materials derived from plant feed stocks. These include development of complex enzyme cocktails for the direct enzymatic processing of these materials, or as a gene cassette for expression by recombinant microbes that function to carry out such processes in bioreactors.

Novel glycoside hydrolase enzymes were identified during the course of an activity-based metagenomic screen that was executed to identify genes encoding fibrolytic enzymes present in the metatranscriptome of a bovine ruminal protozoan-enriched cDNA expression library. Of the novel glycoside hydrolase genes identified was a cDNA encoding a gene active against a hemicellulose substrate, xylan. Further, more detailed biochemical analyses have been performed and indicated that the cDNA (named the Type 2-8.6 cDNA) encodes a novel and highly active arabinoxylanase, which is proven to be highly valuable in various biofuel-related industrial processes.

The rumen, the foregut of herbivorous ruminant animals (e.g., cattle), is a complex ecosystem that functions as a bioreactor to effect processing of complex plant material. Among the numerous and diverse microbes involved in ruminal digestion are the ruminal protozoans, which are single-celled, ciliated eukaryotic organisms. We executed an activity-based screen to identify genes encoding fibrolytic enzymes present in the metatranscriptome of a bovine ruminal protozoan-enriched cDNA expression library. Of the four novel genes identified, two were characterized in biochemical assays. Our results provide evidence for the effective use of functional metagenomics to retrieve novel enzymes from microbial populations that cannot be maintained in axenic cultures.

Therefore, to investigate the potential diversity of fibrolytic enzymes in a total ciliate population, we conducted an activity-based metagenomics screen of the meta-transcriptome of protozoa in the rumen fluid derived from a single, fistulated cow.

Recombinant DNAs comprising the novel glycoside hydrolase enzymes (glycosidase) enzymes are provided herein. In certain embodiments, the recombinant nucleic acids can comprise a heterologous promoter that is operably linked to a gene encoding any one of:
i) a protein having at least 85%, 90%, 95%, 98%, 99% or 100% amino acid sequence identity to SEQ ID NO: 6 and glycosidase activity;
ii) a protein having at least 85% 90%, 95%, 98%, 99% or 100% amino acid sequence identity to SEQ ID NO: 8 and glycosidase activity;
iii) a protein having at least 70%, 75%, 80%, 85%, 90%, 95%, 98%, 99% or 100% amino acid sequence identity to SEQ ID NO: 10 and glycosidase activity; iv) a protein having at least 75% amino acid sequence identity to SEQ ID NO: 12 and glycosidase activity; or,
v) a deletion derivative of a protein having at least 75% amino acid sequence identity to SEQ ID NO: 12 and glycosidase activity.

Recombinant DNAs provided herein can comprise DNA or RNA molecules. In certain embodiments, the promoter provides for expression of the glycosidase in a bacterial cell, a yeast cell, a plant cell, a fungal cell, an algal cell, a protozoan cell, or a mammalian cell. In certain embodiments where the recombinant DNA encodes a protein having at least 85% amino acid sequence identity to SEQ ID NO: 6 and glycosidase activity, the glycosidase can comprises at least one GH5 domain. In certain embodiments where the recombinant DNA encodes a protein having at least 85% amino acid sequence identity to SEQ ID NO: 6 and glycosidase activity, the glycosidase activity comprises a xyloglucanase activity. In certain embodiments where the recombinant DNA encodes a protein having at least 85%, 90%, 95%, 98%, 99%, or 100% amino acid sequence identity to SEQ ID NO: 8 and glycosidase activity, the glycosidase can comprises at least one GH10 domain. In certain embodiments where the recombinant DNA encodes a protein having at least 85% amino acid sequence identity to SEQ ID NO: 8 and glycosidase activity, the glycosidase activity can comprise an arabinoxylanase activity. In certain embodiments where the recombinant DNA encodes a protein having at least 70% amino acid sequence identity to SEQ ID NO: 10 and glycosidase activity, the glycosidase can comprise at least two GH11 domains. In certain embodiments where the recombinant DNA encodes a protein having at least 75% amino acid sequence identity to SEQ ID NO: 12 and glycosidase activity, the glycosidase can comprise at least two GH11 domains. Also provided herein are transformed cells comprising any of the aforementioned recombinant nucleic acids. In certain embodiments, the transformed cell is a bacterial cell, a yeast cell, an algal cell, a protozoan cell, a plant cell, a fungal cell, or a mammalian cell. In certain embodiments, the promoter provides for constitutive and/or inducible expression of the protein in the cell.

Also provided are methods of making a glycosidase comprising the steps of:
a. culturing the transformed cell comprising any of the aforementioned recombinant nucleic acids under conditions that provide for accumulation of the protein in the cell or in the cell culture medium; and,
b. harvesting said protein from said cell or said cell culture medium.

Also provided herein are methods for degrading lignocellulosic, cellulosic, and/or hemicellulosic materials with any of the aforementioned transformed cells. In certain embodiments, a method of degrading lignocellulosic, cellulosic, and/or hemicellulosic materials can comprise culturing the transformed cell in the presence of lignocellulosic, cellulosic, and/or hemicellulosic materials under conditions that provide for accumulation of the protein in the cell or in the cell culture medium and for at least partial hydrolysis of lignocellulosic, cellulosic, and/or hemicellulosic materials. In certain embodiments, a method of degrading hemicellulose can comprise culturing the transformed cell in the presence of hemicellulose under conditions that provide for accumulation of the protein in the cell or in the cell culture medium and for at least partial hydrolysis of xyloglucans in said hemicellulose. In certain embodiments, the cell that provides for at least partial hydrolysis of xyloglucans is a cell transformed with a recombinant nucleic acid encoding a protein having at least 85%, 90%, 95%, 98%, 99% or 100% amino acid sequence identity to SEQ ID NO: 6 and glycosidase activity. In certain embodiments, a method of degrading hemicellulose comprising culturing the transformed cell in the presence of hemicellulose under conditions that provide for accumulation of the protein in the cell or in the cell culture medium and for at least partial hydrolysis of arabinoxyloglucans in said hemicellulose. In certain embodiments, the cell that provides for at least partial hydrolysis of arabinoxyloglucans is a cell transformed with a recombinant nucleic acid encoding a protein having at least 85%, 90%, 95%, 98%, 99%, or 100% amino acid sequence identity to SEQ ID NO: 8 and glycosidase activity. In certain embodiments, the lignocellulose, cellulose, and/or hemicellulose is obtained from plant biomass. In certain embodiments, the plant biomass is selected from the group consisting of corn fiber, corn stover, wheat straw, rice straw, rice bran, switchgrass, wood, and sugarcane bagasse.

Also provided herein are isolated proteins encoded by any of the aforementioned recombinant nucleic acids. Isolated proteins provided herein include:
i) a protein having at least 85%, 90%, 95%, 98%, 99% or 100% amino acid sequence identity to SEQ ID NO: 6 and glycosidase activity;
ii) a protein having at least 85% 90%, 95%, 98%, 99% or 100% amino acid sequence identity to SEQ ID NO: 8 and glycosidase activity;
iii) a protein having at least 70%, 75%, 80%, 85%, 90%, 95%, 98%, 99% or 100% amino acid sequence identity to SEQ ID NO: 10 and glycosidase activity;
iv) a protein having at least 75% amino acid sequence identity to SEQ ID NO: 12 and glycosidase activity; or,
v) a deletion derivative of a protein having at least 75% amino acid sequence identity to SEQ ID NO: 12 and glycosidase activity.

Also provided herein are methods for degrading lignocellulosic, cellulosic, and/or hemicellulosic materials with any of the glycosidases encoded by the aforementioned recombinant DNAs. In certain embodiments, methods for degrading lignocellulosic, cellulosic, and/or hemicellulosic materials comprising incubating any of:
i) a protein having at least 85%, 90%, 95%, 98%, 99% or 100% amino acid sequence identity to SEQ ID NO: 6 and glycosidase activity;
ii) a protein having at least 85% 90%, 95%, 98%, 99% or 100% amino acid sequence identity to SEQ ID NO: 8 and glycosidase activity;
iii) a protein having at least 70%, 75%, 80%, 85%, 90%, 95%, 98%, 99% or 100% amino acid sequence identity to SEQ ID NO: 10 and glycosidase activity;
iv) a protein having at least 75% amino acid sequence identity to SEQ ID NO: 12 and glycosidase activity; or,
v) a deletion derivative of a protein having at least 75% amino acid sequence identity to SEQ ID NO: 12 and glycosidase activity,
   with lignocellulosic, cellulosic, and/or hemicellulosic materials in a reaction vessel under conditions that provide for at least partial hydrolysis of lignocellulosic, cellulosic, and/or hemicellulosic materials is provided. In certain embodiments, methods for degrading hemicellulose comprising incubating a protein having at least 85% identity to SEQ ID NO: 6 and glycosidase activity with hemicellulose in a reaction vessel under conditions that provide for at least partial hydrolysis of xyloglucans in said hemicellulose are provided. In certain embodiments, the conditions comprise a temperature range of about 4° C. to about 60° C. In certain embodiments, the conditions comprise a temperature range of about 35° C. to about 60° C. In certain embodiments, the conditions comprise a pH of about 5.0 to about 8.0. In certain embodiments, the lignocellose, cellulose, or hemicellulose is obtained from plant biomass, paper pulp, or municipal waste. In certain embodiments, the plant biomass is selected from the group consisting of corn fiber, corn stover, wheat straw, rice straw, switchgrass, wood, and sugarcane bagasse. In certain embodiments, the protein of (i), (ii), (iii), (iv), or (v) is an isolated protein.

### DESCRIPTION OF THE DRAWINGS

FIGS. 1A, B, and C illustrate a summary of metagenomic screen positives. (A) Overall domain organization of the polypeptide encoded by the longest cDNA of each Type. The numbers adjacent to the diagrams indicate amino acid residues. The Type 1 cDNA (SEQ ID NO:5) encodes a protein (SEQ ID NO:6) with a single, N-terminal GH5 domain, and a C-terminal domain of unknown function. The Type 2 cDNA (SEQ ID NO:7) encodes a protein (SEQ ID NO:8) with a single GH10 domain. The Type 3 (partial) cDNA (SEQ ID NO:9) encodes a protein (SEQ ID NO:10) with a partial, N-terminal GH11 domain, and second, C-terminal GH11 domain; whereas the Type 4 cDNA (SEQ ID NO:11) encodes a protein (SEQ ID NO:12) with two GH11 domains. (B) Alignment between the Types 3 and 4 protein sequences, with the GH11 domains indicated by boxes. (C) Domain comparison between the Types 3 and 4 proteins. “Domain I” and “Domain II” refer to the first and second GH11 domains, as indicated in (B). The first number indicates percent identity; whereas the second number (in parentheses) indicate percent similarity. “Inter” refers to the interdomain sequence.

FIG. 2 illustrates the phylogenetic topology of rumen protozoan and bacterial glycoside hydrolases. A majority-ruled parsimony tree with maximum-likelihood branch lengths was calculated using full-length amino acids sequences. Bootstrap values of 1000 independent trees larger than 60 were labeled on each branch. Major clades are delimited by solid horizontal lines. Sequences identified in this study are shown in white in a black box. Protozoan sequences are shown in bold italic. GenBank accession numbers for each sequence are given within parenthesis. The Genbank accession numbers and SEQ ID NO (in parentheses) for the corresponding cDNA sequences are as follows: Type 1 (JN635693; SEQ ID NO:5), Type 2 (JN635694; SEQ ID NO:7), Type 3 (JN635695; SEQ ID NO:9) and Type 4 (JN635696; SEQ ID NO:11).

FIG. 3 illustrates substrate specificity analysis for type 1-7.1 and type 2-8.6 recombinant proteins. Substrate specificity assays were performed in triplicate in 250 μL of 1% polysaccharide solutions buffered with either 50 mM MES, pH 6.0 (optimum conditions for Type 1-7.1), or 50 mM MOPS, pH 7.4 (optimum conditions for Type 2-8.6). Reactions were initiated by adding 10 μL (22 g protein/mL) of purified enzyme.

FIG. 4 illustrates HPLC analysis for cleavage product analysis type 1-7.1 and type 2-8.6 recombinant proteins. HPLC chromatographs of hydrolysis products of β-glucan after exposure to 1-7.1 (B) and xylan after exposure to 2-8.6 (D) for 60 minutes. Polysaccharides, β-glucan (A) and xylan (C) without addition of enzyme, were used as controls. The glucose and xylose standard peaks were detected at 4 minutes (data not shown).

FIG. 5 illustrates analysis of Type 4 deletion cDNAs. In addition to 49 intact Type 4 cDNAs, we also recovered eight apparent deletion Type 4 cDNAs. As described below, sequencing of these clones indicated that each of the clones was missing a precise segment of the Type 4 cDNA sequence. While it is possible that these deletion-class cDNAs represent novel gene forms derived from particular species/variant populations within the rumen, the fact that 3-prime untranslated sequences of these cDNAs were essentially identical, suggests that they are cloning artifacts. The eight cDNAs are noteworthy due to the nature of their deletions: each of the seven variants appears to be missing 753 bp, which corresponds to a coding capacity of 251 amino acid residues. This interval precisely corresponds to the sequence lengths of inter-domain region (68 amino acids), plus the length of a single GH10 domain (183 amino acids). The 5-prime breakpoint of each deletion lies in the N-terminal GH10 domain; whereas the 3-prime breakpoint of each deletion lies in the second GH10 domain. Because the ‘window’ of the deletions is located at a unique position in each cDNA, each clone encodes a unique Domain I/Domain II hybrid; because they were identified through activity-based screening procedures, the hybrids must encode proteins with some activity toward the xylan substrate. Examination of the breakpoint in the context of a DNA sequence alignment between the two GH10 domains in the full-length Type 4 cDNA (data not shown), revealed that each occurred within or adjacent to a stretch of conserved sequence shared between the two domains. Because the cDNA sequences outside the deletions were well conserved, even in the 3-UTR of the cDNAs, we hypothesize that these aberrant, shortened cDNA forms likely arose during cloning by a homologous recombination mechanism.

FIG. 6 illustrates the purification of recombinant Type 1-7.1 protein. Lane 1: Cell pellet, post-induction. Lane 2: Cell Pellet, pre-induction. Lanes 3 and 6: Invitrogen BenchMark Pre-stained Molecular Weight Markers; apparent masses indicated in kDa. Lanes 5 and 6: Post-affinity purification samples of recombinant Type 1-7.1 protein.

FIGS. 7A, B, C, D illustrates pH and temperature tolerances for Type 1-7.1 enzyme. Effect of pH on activity of Type 1-7.1 towards AZCL-HE-cellulose; 1 mg of AZCL-HE-cellulose was suspended in Britton-Robinson buffers (Britton and Robinson, 1931), incubation at 37° C. for one hour, release of AZCL measured by absorbance at 590 nm (A). More precise pH tolerance of Type 1-7.1 was determined by monitoring release of reducing sugars (Anthon and Barrett, 2002) from 1% CMC in 50 mM sodium acetate buffer, incubated at 37° C. for one hour (B). Temperature tolerance was determined by monitoring release of reducing sugars (Anthon and Barrett, 2002) from 0.1% β-glucan in 50 mM MES, pH 6 buffer, incubated at 4° C., 24° C., 37° C., 50° C., and 60° C., in triplicate, for one hour (C); and incubated at 40.0° C., 42.2° C., 46.7° C., 52.1° C., 57.1° C., and 60.0° C., in triplicate, for one hour (D).

FIG. 8 A, B, C illustrates the pH and temperature tolerances for Type 2-8.6 enzyme. Effect of pH on activity of Type 1-7.1 towards AZCL-xylan; 1 mg of AZCL-xylan was suspended in Britton-Robinson buffers (Britton and Robinson, 1931), incubated at 37° C. for one hour, release of AZCL was measured by absorbance at 590 nm (A). More precise pH tolerance of Type 2-8.6 was determined by monitoring release of reducing sugars (Anthon and Barrett, 2002) from 1% xylan in 50 mM MOPS buffer at pH values 6.8-7.8, incubated at 37° C. for 15 minutes in triplicate (B). Temperature tolerance was determined by monitoring release of reducing sugars (Anthon and Barrett, 2002) from 1% xylan in 50 mM MOPS, pH 7.4 buffer, incubated at 26° C., 30° C., 35° C., 40° C., 45° C., 50° C., 55° C., and 60° C., in triplicate, for 15 minutes (C).

FIG. 9 illustrates a comparison of the Type 1 protein sequence of SEQ ID NO:6 (gi 349734011) to a pfam00150 consensus sequence of SEQ ID NO: 15.

### DESCRIPTION

### Definitions

As used herein, the term “heterologous”, when used in the context of two nucleic acid or protein sequences, refers to sequences that not contiguous to one another in nature. For example, a yeast secretion signal peptide sequence and a protozoan polypeptide sequence are heterologous because the two sequences are not naturally contiguous.

As used herein, the phrase “isolated protein” refers to a protein that has been separated from its naturally occurring cellular host.

As used herein, the term “glycosidase” refers to an enzyme that can hydrolyse at least one substrate in a group comprising lignocelluloses, celluloses, hemicelluloses, xylan, beta-glucan, carboxymethylcellulose, arabinoxylan, xyloglucan, and derivatives thereof. The term “glycosidase” and the phrase “glycosyl hydrolase” are used interchangeably herein.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide for protein localization functions (signal peptides for extracellular secretion, organellar targeting peptides, and the like), inteins, sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

Various recombinant nucleic acids encoding glycosidases useful for degradation of lignocellulose, cellulose, and hemicellulose are provided herein. In certain embodiments, the recombinant nucleic acids can comprise a heterologous promoter that is operably linked to a gene encoding any one of:
vi) a protein having at least 85%, 90%, 95%, 98%, 99% or 100% amino acid sequence identity to SEQ ID NO: 6 and glycosidase activity;
vii) a protein having at least 85% 90%, 95%, 98%, 99% or 100% amino acid sequence identity to SEQ ID NO: 8 and glycosidase activity;
viii) a protein having at least 70%, 75%, 80%, 85%, 90%, 95%, 98%, 99% or 100% amino acid sequence identity to SEQ ID NO: 10 and glycosidase activity;
ix) a protein having at least 75% amino acid sequence identity to SEQ ID NO: 12 and glycosidase activity; or,
x) a deletion derivative of a protein having at least 75% amino acid sequence identity to SEQ ID NO: 12 and glycosidase activity.

While the recombinant nucleic acids will typically comprise DNA molecules, the use of recombinant RNAs including, but not limited to, viral RNA vectors is also provided herein. Such viral vectors can comprise a promoter recognized by an RNA-dependent RNA polymerase that is operably linked to an RNA encoding any one of the aforementioned proteins. In certain embodiments, the glycosidase encoding sequence can also be operably linked to a sequence encoding a signal peptide that provides for secretion of the glycosidase from a cell. Such signal peptides can be from a heterologous organism. In certain embodiments, the glycosidase encoding sequence can also be operably linked to a sequence encoding a polyadenylation site and/or transcription termination sequence. Useful polyadenylation site and/or transcription termination sequences can be obtained from a homologous or heterologous source.

Useful promoters that can be used in the recombinant DNAs include promoters that provide for expression of the glycosidase in a bacterial cell, a yeast cell, a plant cell, a fungal cell, an algal cell, a protozoan cell, or a mammalian cell. In certain embodiments, the promoter can be an inducible promoter. Exemplary and non-limiting bacterial promoters include, but are not limited to, bacteriophage, pTAC, pLAC, and pARA (arabinose inducible) promoters. Methanol inducible promoters can also be used in the recombinant DNA vectors. In certain embodiments, the methanol inducible promoters can comprise an AOX promoter (Alcohol Oxidase promoter), DHAS promoter (or DAS promoter) (dihydroxyacetone synthase promoter), FDH promoter (or FMDH promoter) (formate dehydrogenase promoter), MOX promoter (Methanol Oxidase promoter), ZZA1, PEX5-, PEX8-, and PEX14-promoters. Exemplary and non-limiting methanol inducible promoters also include, but are not limited to, promoters from yeast such as Pichia, Hansenula, Candida, and Torulopsis (U.S. Pat. Nos. 8,143,023, 5,750,372 and 6,001,590).

In certain embodiments, the encoded glycosidase protein will comprise at least one conserved protein sequence domain that is characteristic of proteins belonging to certain glycosyl hydrolase superfamily. Recombinant nucleic acids encoding proteins having at least 85% amino acid sequence identity to SEQ ID NO: 6 and glycosidase activity can thus comprise at least one

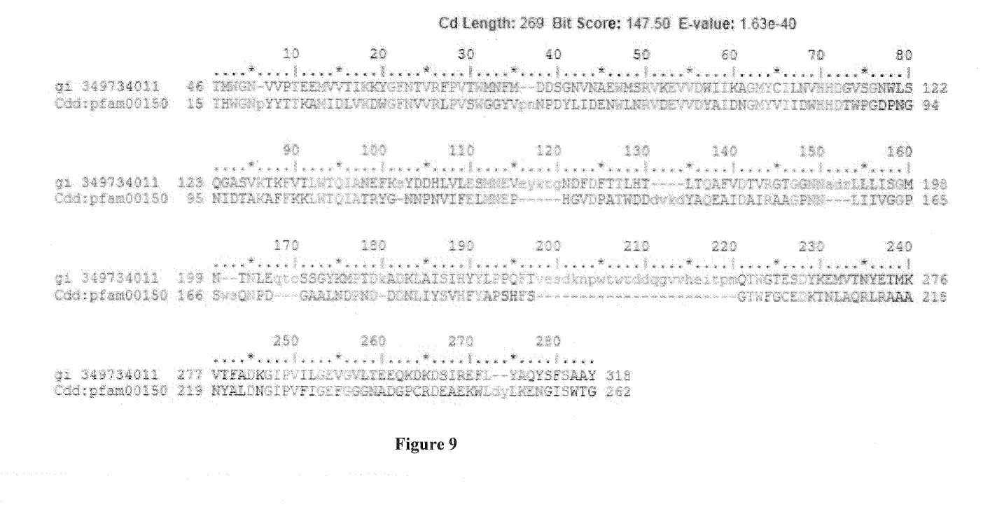

GH5 domain (i.e. a glycosyl hydrolase family 5 domain). A comparison between the SEQ ID NO:6 protein and a pfam00150 consensus sequence (SEQ ID NO: 18) that shows conserved sequence motifs is provided in FIG. 9. Other glycosidase proteins containing GH5 domains have been described (26, 27; and also “pfam00150” on the World Wide Web (internet) at “ncbi.nlm.nih.gov/Structure/cdd/cddsrv.cgi?uid=201037”). The pfam database of conserved protein domains has also been described by Marchler-Bauer A et al. (2011), “CDD: a Conserved Domain Database for the functional annotation of proteins”, Nucleic Acids Res. 39(D)225-9. Structure-function relationships where certain amino acid residues in GH5, GH8, GH10, and GH11 containing cellulases are implicated in xylanase substrate specificity have also been reported (Pollet et al. Crit. Rev Biotechnol. 2010 September; 30(3):176-91). Catalytic and substrate binding residues in the related E. chrysanthemi GH5 domain containing xylanase have also been identified (Larson et al., Biochemistry 42: 8411-8422, 2003). In certain embodiments, residues in a GH5 domain of the SEQ ID NO:6 protein that are involved in catalysis and/or substrate specificity will be retained in proteins having at least 85%, 90%, 95%, 98%, 99% or 100% amino acid sequence identity to SEQ ID NO: 6 and glycosidase activity. In certain embodiments, conserved motifs in a GH5 domain are be retained in proteins having at least 85%, 90%, 95%, 98%, 99% or 100% amino acid sequence identity to SEQ ID NO: 6 and glycosidase activity. In certain embodiments, the conserved motifs retained in the GH5 motif of the protein having at least 85%, 90%, 95%, 98%, 99% or 100% amino acid sequence identity to SEQ ID NO: 6 and glycosidase activity can comprise at least one of a “RV (K/D)EVVD” motif (SEQ ID NO:16), a “LWTQIA” motif (SEQ ID NO:17), and/or a “D(K/N)GIPV(I/F)(L/I)GE(V/F)G” (SEQ ID NO: 18) motif. In certain embodiments, the glycosidase activity of proteins having at least 85%, 90%, 95%, 98%, 99% or 100% sequence identity to SEQ ID NO:6 can comprise a preference for xyloglucan substrates. In certain embodiments, the glycosidase activity of proteins having at least 85% sequence identity to SEQ ID NO:6 can exhibit specific activities of at least about 500, 600, 700, or 800 Units/mg to about 900 or 1,000 Units/mg towards xyloglucan substrates. In certain embodiments, the glycosidase activity of proteins having at least 85% sequence identity to SEQ ID NO:6 can exhibit specific activities of at least about 500, 600, 700, or 800 Units/mg towards xyloglucan substrates.

Also provided are proteins having at least 85%, 90%, 95%, 98%, 99% or 100% amino acid sequence identity to SEQ ID NO: 8 and glycosidase activity that comprise at least one GH10 domain. Other glycosidase proteins containing GH10 domains have been described (26, 27; Pollet et al. 2010, Ibid.; and also “pfam00331” on the World Wide Web (internet) at “ncbi.nlm.nih.gov/Structure/cdd/cddsrv.cgi?uid=201160”). In certain embodiments, the glycosidase activity of proteins having at least 85% sequence identity to SEQ ID NO:8 can comprise a preference for arabinoxylan substrates. In certain embodiments, the glycosidase activity of proteins having at least 85% sequence identity to SEQ ID NO:6 can exhibit specific activities of at least about 300, 400, 500 to about 600 or 700 Units/mg towards arabinoxylan substrates.

Also provided are proteins having at least 70%, 75%, 80%, 85%, 90%, 95%, 98%, 99% or 100% amino acid sequence identity to SEQ ID NO: 10 and glycosidase activity that comprise at least two GH11 domains. Other glycosidase proteins containing GH11 domains have been described (26, 27; Pollet et al. 2010, Ibid.; and also “pfam00457” on the World Wide Web (internet) at “ncbi.nlm.nih.gov/Structure/cdd/cddsrv.cgi?uid=201240”). In certain embodiments, the glycosidase activity of proteins having at least 70% sequence identity to SEQ ID NO:10 can comprise activity towards xylan substrates.

Also provided are proteins having at least 75%, 80%, 85%, 90%, 95%, 98%, 99%, or 100% amino acid sequence identity to SEQ ID NO: 12 and glycosidase activity that comprise at least two GH11 domains. Other glycosidase proteins containing GH11 domains have been described (26, 27; Pollet et al. 2010, Ibid.; and also “pfam00457” on the World Wide Web (internet) at “ncbi.nlm.nih.gov/Structure/cdd/cddsrv.cgi?uid=201240”). In certain embodiments, the glycosidase activity of proteins having at least 75% sequence identity to SEQ ID NO:12 can comprise activity towards xylan substrates.

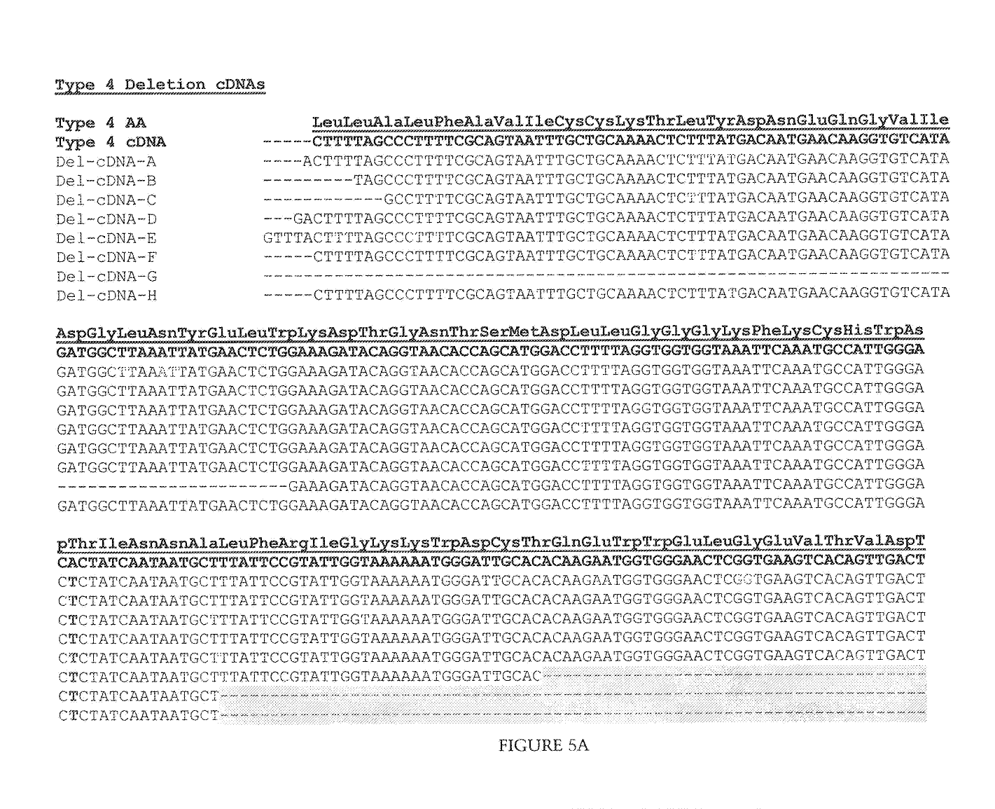

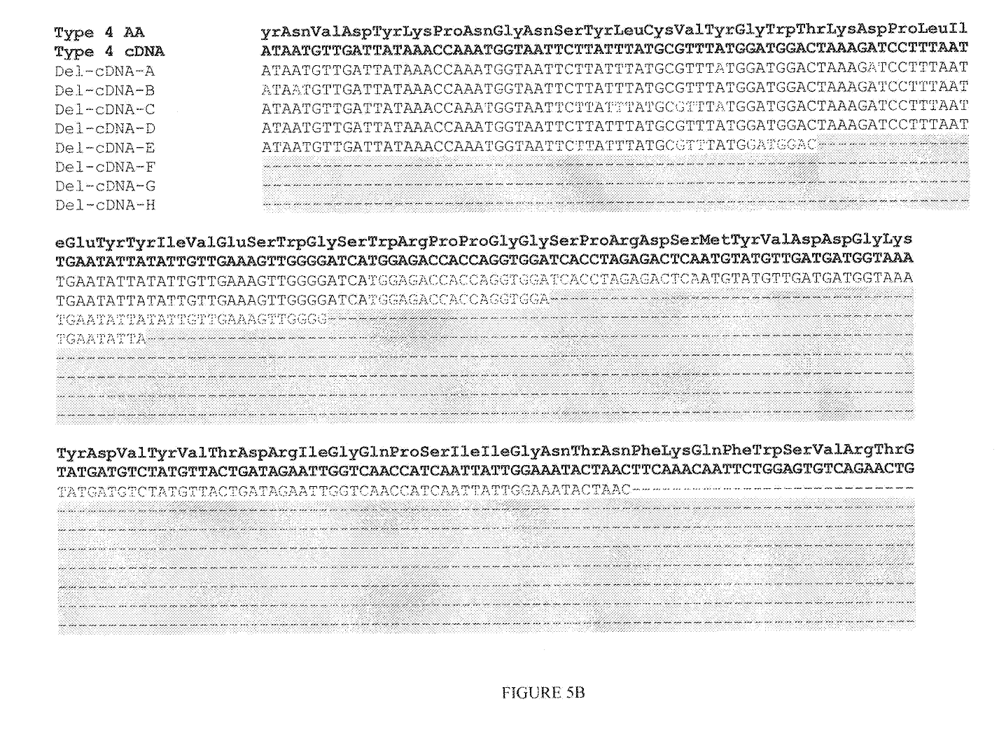

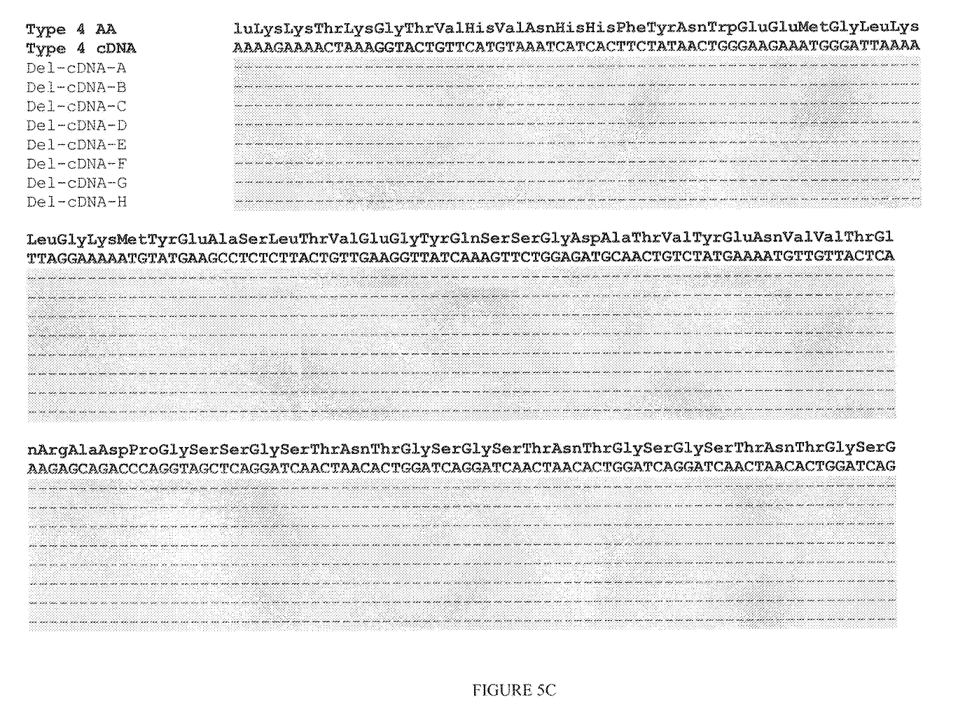

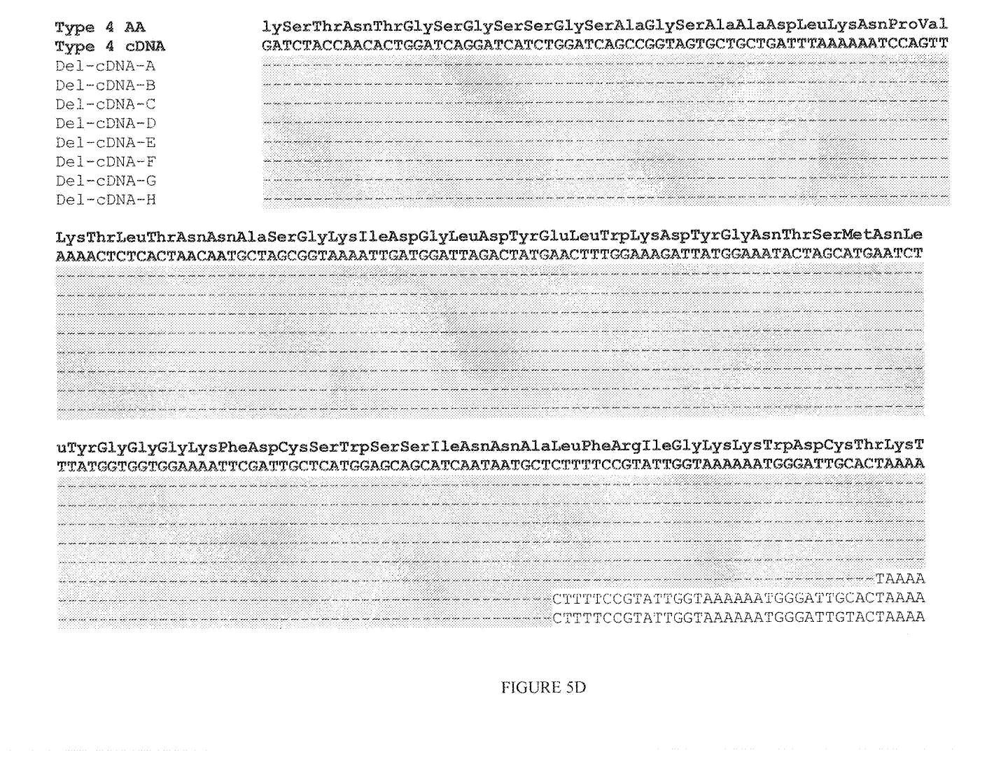

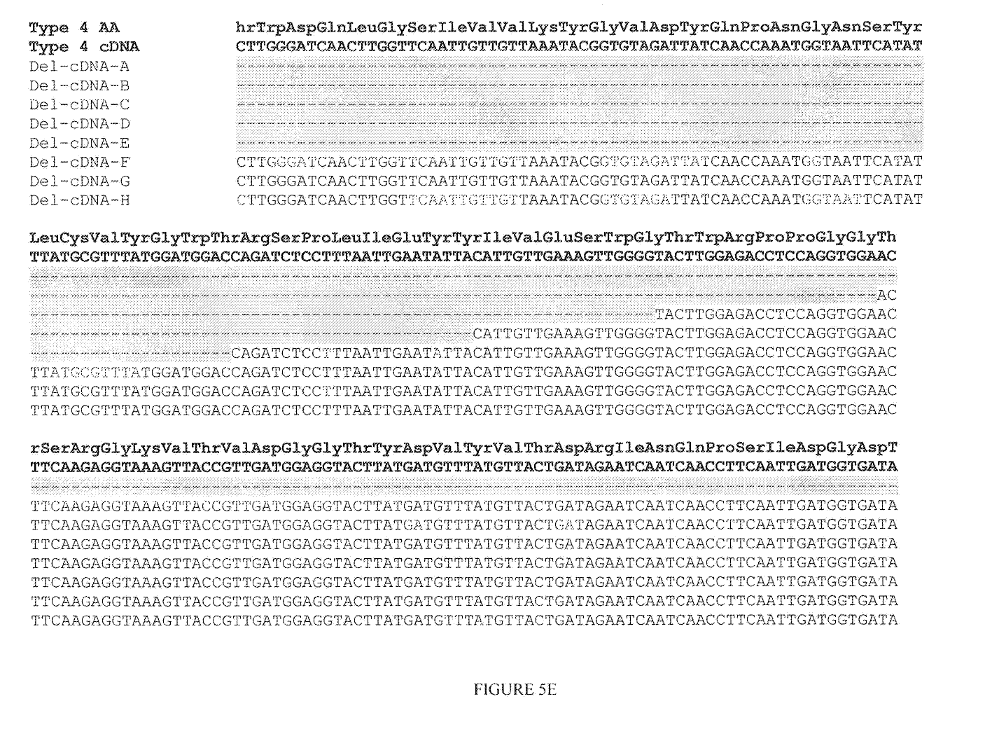

Also provided are deletion derivatives of a protein having at least 75% amino acid sequence identity to SEQ ID NO: 12 and glycosidase activity. In certain embodiments, these deletion derivatives can comprise any of the deletion type Type 4 cDNAs disclosed in FIG. 5 that exhibit activity towards a xylan substrate, cells transformed with those recombinant DNAs, and related methods of making and using glycosidases encoded by the deletion type Type 4 cDNAs. Recombinant DNAs comprising promoters that are operably linked to any one of Del-cDNA-A, Del-cDNA-B, Del-cDNA-C, Del-cDNA-D, Del-cDNA-E, Del-cDNA-F, Del-cDNA-G, and Del-cDNA-H of FIG. 5 are thus provided. In certain embodiments, the deletion derivative of SEQ ID NO:12 is a protein encoded by a cDNA selected from the group consisting of Del-cDNA-A, Del-cDNA-B, Del-cDNA-C, Del-cDNA-D, Del-cDNA-E, Del-cDNA-F, Del-cDNA-G, and Del-cDNA-H of FIG. 5.

A variety of transformed cells containing the recombinant nucleic acid are also provided herein. In certain embodiments, the transformed cell can be a bacterial cell, a yeast cell, an algal cell, a protozoan cell, a plant cell, a fungal cell, or a mammalian cell. Transformed microorganisms that are particularly useful or adapted for degradation of lignocellulosic, hemicellulosic, and/or cellulosic materials in bioreactors are specifically contemplated. In certain embodiments, the transformed microorganism is a bacterium. Bacteria that can be transformed with the recombinant nucleic acids provided herein include, but are not limited to, Escherichia, Zymomonas, Streptomyces, Bacillus, Lactobacillus, Thermoanaerobacterium, and Clostridium species. In another embodiment, the recombinant nucleic acids can be used to transform Escherichia coli, Zymomonas mobilis, Bacillus stearothermophilus, or Clostridia thermocellum. In certain embodiments, the transformed microorganism is a yeast. Yeasts that can be transformed with the recombinant nucleic acids provided herein include, but are not limited to, Saccharomyces, Schizosaccharomyces, Pichia, Hansenula, Candida, Rhodotorula, Kluyveromyces, and Torulopsis species. In certain embodiments, the transformed microorganism is a fungal microorganism. Fungal microorganism that can be transformed with the recombinant nucleic acids provided herein include, but are not limited to, Aspergillus, Trichoderma, Rhizopus, and Mucor species. In certain embodiments, the transformed microorganism is an algal microorganism. Algal microorganisms that can be transformed with the recombinant nucleic acids provided herein include, but are not limited to, Thraustochytrium and Schizochytrium species (Cheng et al. Microbiol. Res. 2012 Mar. 20; 167(3):179-86; US Patent Application Publication No. US20110086390). In certain embodiments, the transformed microorganism is a protozoan microorganism. Protozoan microorganisms that can be transformed with the recombinant nucleic acids provided herein include, but are not limited to, Tetrahymena species.

Transformed plant cells containing the recombinant nucleic acids and transformed plants comprising those recombinant nucleic acids are also provided herein. In certain embodiments, the recombinant nucleic acid will provide for regulated induction or activation of the encoded glycosidase protein on an as needed or as desired basis. In certain embodiments, the encoded glycosidase can be interrupted by, or fused to single or multiple Controllable InterVening Protein Sequence (CIVPS) or intein sequences. Controllable InterVening Protein Sequence (CIVPS) or intein sequences and their use in transgenic plants are described in U.S. Pat. No. 8,247,647, which is incorporated herein by reference in its entirety. Other compositions and methods for inducing or activating an enzyme that can be adapted to the glycosidases provided herein are disclosed in U.S. Pat. No. 7,102,057, which is also incorporated herein by reference in its entirety. Plant cells and plants that can be transformed with recombinant nucleic acids provided herein include, but are not limited to, corn, soy, cotton, rice, wheat, sorghum, sugarcane, switchgrass, poplar, aspen, coniferous plants, and the like.

Also provided herein are methods of using the glycosidases, or transformed microorganisms comprising recombinant nucleic acids encoding any of the glycosidases, to degrade lignocellulosic materials or derived cellulosic and/or hemicellulosic materials. Various methods for obtaining cellulosic and/or hemicellulosic materials from lignocellulosic material present in biomass have been described in the literature. Pre-treatment of lignocellulosic materials to render such material suitable for subsequent enzymatic hydrolysis of cellulose and hemicellulose components include, but are not limited to, concentrated acid, dilute acid, alkaline, sulfite, hydrogen peroxide, steam explosion (autohydrolysis), ammonia fiber explosion (AFEX), wet-oxidation. lime. liquid hot water, carbon dioxide explosion, and organic solvent treatments (see (Saha, J Ind Microbiol Biotechnol (2003) 30: 279-291 and references cited therein). Techniques for obtaining cellulosic and/or hemicellulosic materials suitable for enzymatic digestion from lignocellulosic material present in biomass are also disclosed in U.S. Pat. Nos. 8,173,406, 8,133,393, 8,057,639, 7,998,713, and 5,411,594, each incorporated herein by reference in their entireties.

In methods for degrading lignocellulosic, cellulosic, or hemi-cellulosic materials provided herein, treatment of materials with any of the glycosidases can be supplemented by treatment with additional glycosidases with complementary substrate specificities. In certain embodiments, treatment with any of the glycosidases provided herein can be supplemented with treatment with one or more lignin degrading enzyme(s), cellulose degrading enzyme(s), and/or exoglucanase(s). Such treatments can be achieved either by exposing the lignocellulosic, cellulosic, or hemi-cellulosic materials to glycosidase enzyme preparations. to transformed host cells comprising the glycosidases, or to conditioned cell culture media comprising the glycosidases obtained from the transformed host cells. Supplemental treatment with additional glycosidases with complementary substrate specificities can be either simultaneous with, prior to, or after treatment with the glycosidases provided herein.

### EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Materials and Methods Used

Materials. Carboxymethyl cellulose (CMC), cellulose (fibrous, medium), galactan, laminarin from Laminaria digitata, mannan from Saccharomyces cervisiae, and xylan from beechwood were obtained from Sigma-Aldrich. AZCL-HE-cellulose, AZCL-xylan (oat), arabinan (sugar beet), β-glucan (oat, medium viscosity), wheat arabinoxylan (medium viscosity) and xyloglucan from tamarind seed (amyloid) were obtained from Megazyme. Reagents for the reducing sugar assay, ammonium iron (III) sulfate dodecahydrate, 3-methyl-2-benzothiazolinone hydrazone hydrochloride hydrate, and sulfanic acid were obtained from Sigma-Aldrich. Isopropyl β-D-1-thiogalactopyranoside (IPTG) was obtained from Gold Biotechnology.

Rumen Sample Collection, Protozoan Purification and mRNA Purification. Rumen protozoa were harvested by using a procedure based on (36), modified as follows: approximately 3 L of total rumen content (fluid and solids) were collected from a fistulated Holstein cow maintained at the University of Missouri Dairy Farm, in Columbia, Mo. The donor cow was fed a total mixed ration (TMR) of a common lactation diet, that consisted of alfalfa haylage, corn silage, corn and protein, minerals and fat-soluble vitamins added to meet or exceed nutrient requirements. The rumen sample was collected in the morning, prior to feeding, into a pre-warmed canister, and transported to the lab within 30 minutes. Aliquots of the sample were treated in three pulses, 30 sec each, with a blender, and pressed through a double layer of cheesecloth to remove bulk solids. The resulting liquid (approximately 1 L) was supplemented to 1% maltose and 0.5% sucrose and then floculated anaerobically for 1 hr at 39° C. After aspirating the floating feed particle layer, the liquid was dialyzed against eight changes of 39° C. Coleman anaerobic buffer (55) using a home-made 10-μm pore size NITEX filter cloth (Sefar America) bag with gentle agitation. The volume of the resulting material was −50 mL and represented a concentrated mixture of protozoa, whose composition was verified by microscopic observation. Total RNA was isolated from this material using the TRIzol® Plus RNA Purification System (Invitrogen); mRNA was then purified using the magnetic bead-based FastTrack® 2.0 mRNA Isolation Kit (Invitrogen).

Lambda Zap-Based Protozoan cDNA Library Construction. A Lambda Zap II-based protozoan cDNA expression library was constructed by using the Zap cDNA synthesis kit (Stratagene catalog #200401), starting with 5 μg of polyadenylated mRNA (for detailed protocols, see on the world wide web (internet) site: “genomics.agilent.com/files/Manual/200401.pdf”). After size-fractionation of total cDNA by gel-exclusion chromatography using Sepharose® CL-2B gel filtration medium (procured from Stratagene) in a 1-mL disposable plastic pipet, cDNA fractions ranging from 0.5 to >10 kb were pooled and ligated into the prepared lambda vector (see below), and packaged using the ZAP-cDNA® Gigapack® III Gold Cloning Kit (Stratagene catalog number 200450), according to protocols provided by the manufacturer. After titering, 700,000 p.f.u. of the primary library were amplified on plates using standard lambda phage procedures (Stratagene Zap cDNA Synthesis manual), in order to generate the secondary library, used for activity-based screening.

Activity-based Screening for Fibrolytic Enzymes. To identify candidate fibrolytic enzymes, we utilized IPTG-inducible cDNA expression, plaque-based high-throughput screening on plates containing dye-linked insoluble polysaccharide substrates (44, 51). We screened for two classes of fibrolytic enzymes: xylanases and cellulases, using AZCL-HE-Cellulose and AZCL-Xylan (Oat), respectively (Megazyme, Inc.). The expression library was screened on Petri dishes containing NZYM bottom agar, supplemented with a 1× micronutrient solution (1000×MNS: 3.0 mM H₃BO₃; 0.46 mM MnCl₂; 0.16 mM CuSO₄; 0.6 mM ZnSO₄; 0.1 mM NaMoO₄; 0.01 mM NiSo₄; 0.01 mM CoCl₂), and NZYM top agarose, supplemented with 1×MNS plus 20 mM IPTG. To identify cDNA clones encoding fibrolytic enzymes, the top agarose incorporated either AZCL-HE-Cellulase or AZCL-Xylan (Oat), at a final concentration of 0.3% (w/v). For each substrate, approximately one million p.f.u. were screened (10,000 p.f.u. per 150 mm plate). Over a 2-5 day incubation period at 37° C., 70 clones were picked that exhibited xylan-degrading activity and ten that exhibited cellulose-degrading activity. Positives were plaque-purified in three rounds of plaque purification, and then in vivo-excised to generate pBluescript DNA preparations, using procedures described in the Stratagene Zap cDNA Synthesis manual. To initially characterize the positives, rescued plasmid-borne cDNAs were sequenced with the T3 promoter primer (5′-AAT-TAA-CCC-TCA-CTA-AAG-GG-3′; SEQ ID NO:13), which flanks the 5-prime end of the directionally-cloned cDNA insert. Selected clones of the longer cDNA types (Types 3 and 4, see below), were then completely sequenced with the T7 promoter primer (5′-TAA-TAC-GAC-TCA-CTA-TAG-GG-3′; SEQ ID NO:14) in addition to custom, internal primers, when required (data not shown). To initially assess the diversity of the cDNA collection, we used the CAP3 Sequence Assembly Program (30), BLASTp (1) and ClustalW2 (6).

Sequence Searches, Alignments and Phylogeny.

Protozoan and bacterial glycoside hydrolase sequences were collected for our analysis by BLASTp searches (31) of the GenBank non-redundant protein sequences database (nr), using default search parameters to identify sequences with homology values of 1e-50. Protein sequences were aligned using MUSCLE3.6 (16) with a FASTA output format, and then manually edited using Jalview (7). Majority-ruled parsimonious trees were generated using the program “protpars” of PHYLIP (18), with maximum likelihood branch lengths calculated using TREE-PUZZLE (46). Bootstrap values were calculated using the program “seqboot” of the PHYLIP package. All trees were viewed and printed into a pdf format using A Tree Viewer (58).

Molecular Cloning for Expression Constructs.

To investigate the biochemical properties of representative positives (see Results section for summary of positive classes and FIG. 5), we focused on a single-domain gene for each substrate screened, a Type 1 (identified on cellulose substrate) and a Type 2 (identified on xylan substrate). The coding sequence of the longest cDNA from each class was cloned into the Nco I and Xho I sites of the multiple cloning site of the C-terminal (His)-6-tag expression vector, pET29a (Novagen). To accomplish this, we added restriction sites to PCR primers, and amplified DNA fragments as follows. For Type I (cellulose substrate), the 5-prime primer sequence was GGG-CCA-TGG-CTT-TGG-GCT-TAA-TTT-CAA-TTTC (SEQ ID NO: 1; NcoI site underlined and the first codon of the cDNA indicated by bold text) and the 3-prime PCR primer sequence is GGG-CTC-GAG-TTT-GGA-AAC-AGC-GGC-TTT-GTA-AG (SEQ ID NO:2; XhoI site underlined). For the Type 2, the 5-prime primer sequence was GGG-CCA-TGG-CTT-TAA-ATT-ATG-TAT-CAT-CTA-ATA-ATT-TTC (SEQ ID NO:3; NcoI site underlined and the first codon of the cDNA indicated by bold text) and the 3-prime PCR primer sequence was GGG-CTC-GAG-TGC-TCC-AGC-AAC-TTG-CAT-AAT (SEQ ID NO:4; XhoI site underlined). CDS sequences were amplified using the Platinum PCR Supermix High-Fidelity Kit (Invitrogen) or Phusion® High-Fidelity DNA Polymerase (New England Biolabs), under conditions recommended by the manufacturers. The resulting amplicon DNAs were purified using the Wizard® PCR DNA Purification System (Promega) and cloned into pET29a prepared by standard molecular biology techniques. Miniprep (Promega) DNA for each Type were sequenced with T7 promoter and T7 terminator primers to verify the absence of PCR-derived mutations. The resulting expression constructs, and proteins derived from them, are hereafter designated Type 1-7.1 and Type 2-8.6.

Expression and Purification of Recombinant Enzymes.

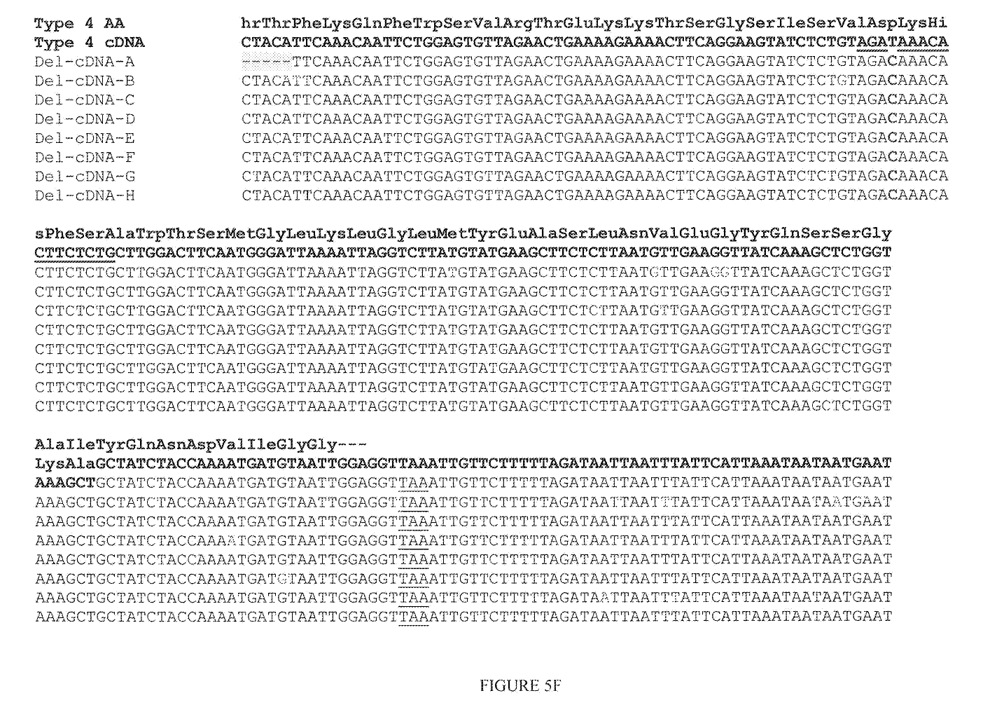

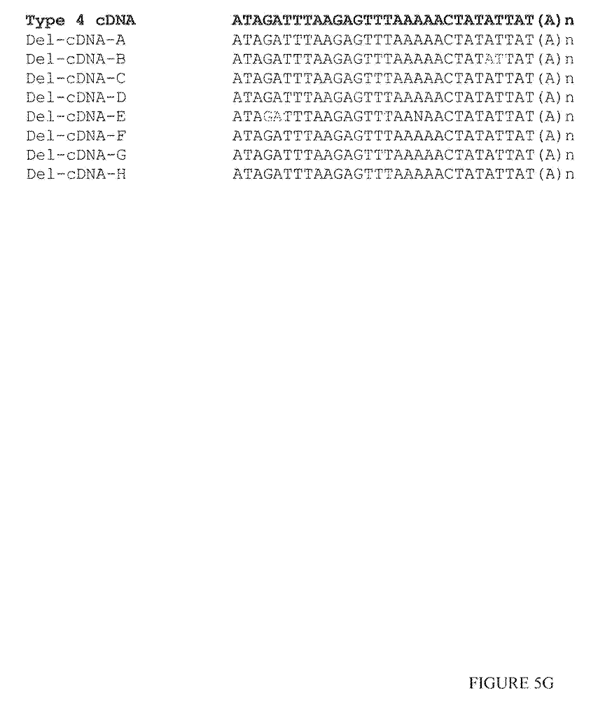

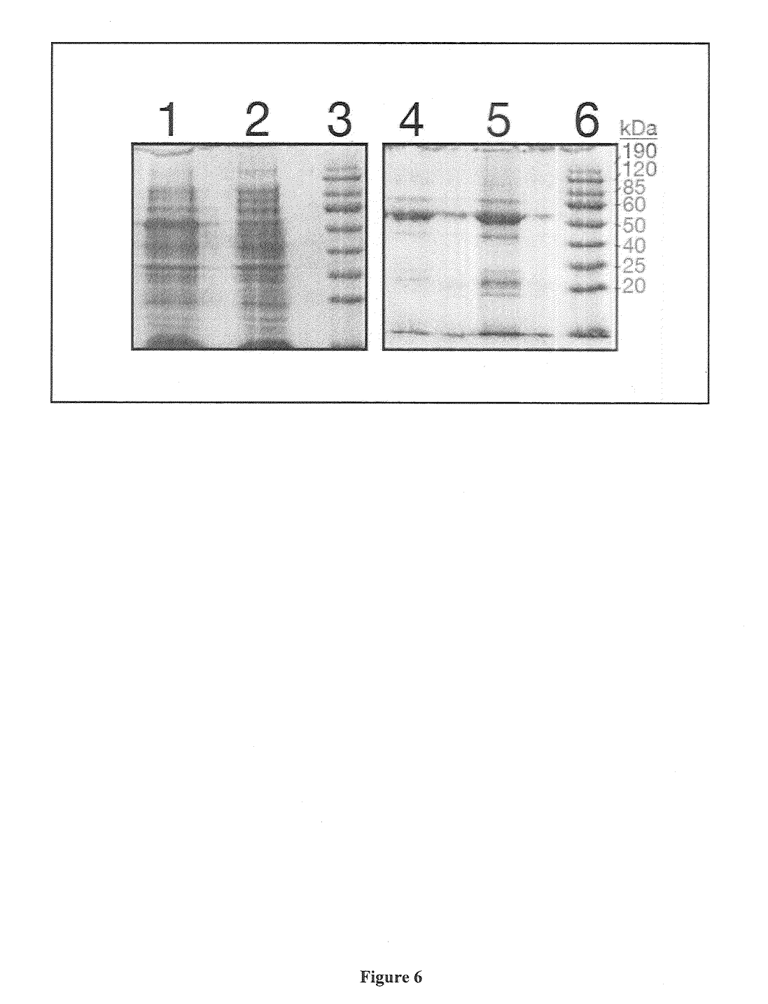

The Type 1-7.1 and Type 2-8.6 pET29a constructs were transformed into E. coli BL21 DE3 cells (Invitrogen), and expression cultures for each construct were grown at 37° C. in 500 mL LB broth containing 30 μg/mL kanamycin. The cultures were grown to an OD₆₀₀ 0.6 to 0.8, at which point expression was induced by the addition of IPTG to a final concentration of 1 mM. Cultures were then grown at 37° C. for an additional three hours. Bacterial cells were harvested by centrifugation (10,000 g at 4° C. for 10 min) and resuspended in 20 mL of equilibration/wash solution (50 mM sodium phosphate buffer, pH 7.0, and 300 mM NaCl) supplemented with 1× Complete®, EDTA-free protease inhibitor cocktail (Roche), and phenylmethylsulphonyl fluoride to a final concentration of 1 mM. Cell resuspensions were lysed using a French press, and the resulting lysates were centrifuged at 10,000 g at 4° C. for 10 min, to remove cell debris. Recombinant proteins were then affinity-purified by TALON® Metal Affinity Resin (Clontech), according to manufacturer's protocols. The purified enzymes were eluted in a single-step elution with equilibration/wash buffer supplemented with 150 mM imidazole. Proteins were then concentrated and imidazole removed with an ultrafiltration membrane (Vivaspin-20 column, GE Healthcare). Enzyme purity was confirmed by SDS-PAGE (see FIG. 6) and protein concentration determined using the Quick Start Bradford Dye Reagent (Biorad).

Enzyme Assays.

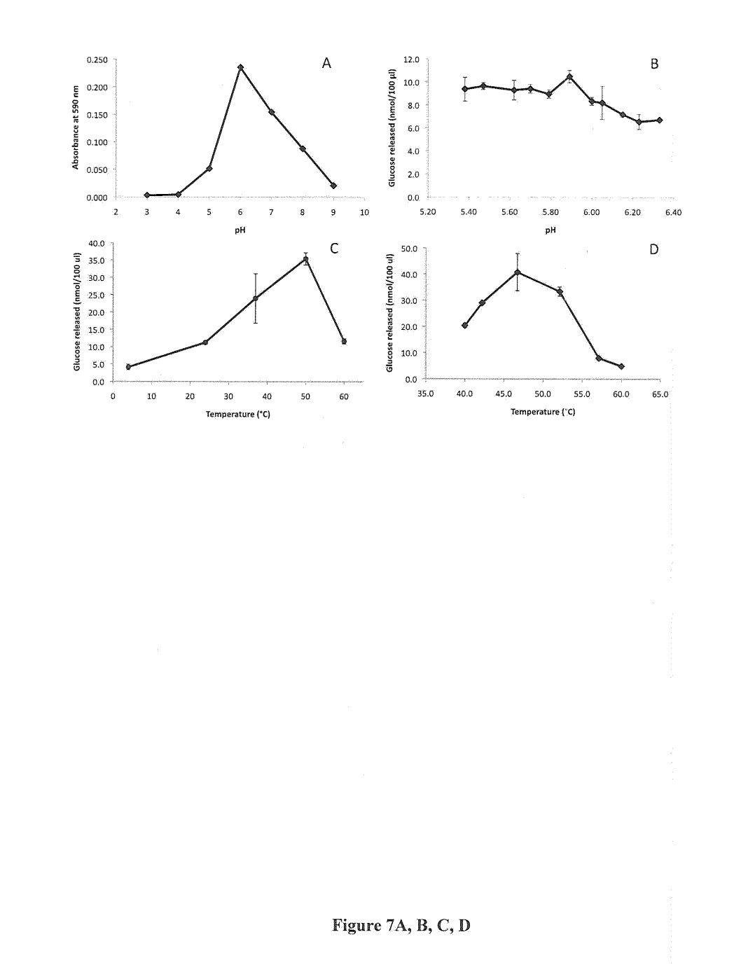

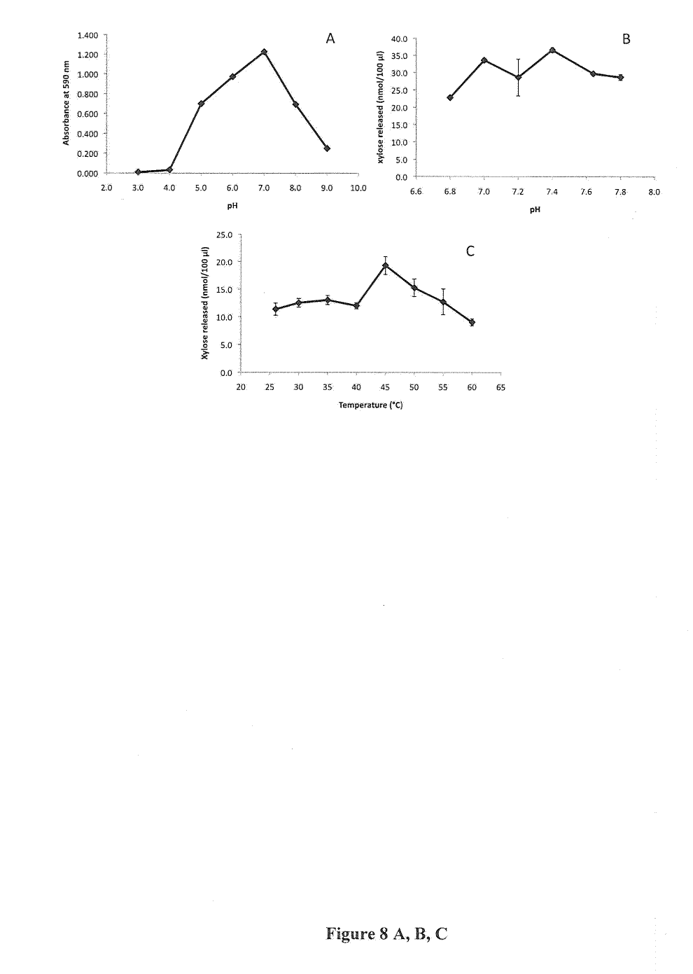

The activity of each enzyme was initially confirmed in a simple, colorimetric assay using the same insoluble substrates that were used in the plate screening procedure. Specifically, one mg of the respective substrate (AZCL-HE-cellulose or AZCL-xylan) was suspended in one mL of protein purification equilibration buffer (300 mM NaCl, 50 mM sodium phosphate, pH 7.0), at 37° C. for 30 min. Reactions were initiated by adding 50 μL of purified enzyme (0.5 to 2.2 mg/mL protein), and the release of solubilized dye was visually validated. Optimal pH conditions were then preliminarily determined in assays using 550 μL of 50 mM Britton-Robinson buffers (5) plus 400 μL 0.2% AZCL-labeled substrates. Reactions were initiated by adding 50 μL of purified enzyme solution (10 μg protein/μL). After incubation at 37° C. for 1 hr, supernatant absorbance at 590 nm was determined (FIGS. 7A and 8A). More precise pH optimal activity determinations were made by measuring the release of reducing sugars from polysaccharides by using the 3-methyl-2-benzothiazolinone hydrazone reagent (MBTH) (2). Assays were run with either 1% CMC in 50 mM sodium acetate buffers or with 1% xylan in 50 mM 3-(N-morpholino) propanesulfonic acid (MOPS) buffer. Assays to determine optimal temperature, substrate specificities, and enzymatic activities were performed in 250 μL of 1% polysaccharide solutions buffered with either 50 mM MES (2-(N-morpholino) ethanesulfonic acid), pH 6.0, or 50 mM MOPS, pH 7.4. Reactions were initiated by adding 10 μL (22 μg protein/mL) of purified enzyme. After incubation, aliquots were added to the MBTH reagent. The quantities of released reducing sugars were determined using glucose, mannose, galactose, arabinose, and xylose as standards. The apparent Kₘ and Vₘₐₓ values were determined by fitting the rate data to the Michaelis-Menten equation (KaleidaGraph, version 3.6, Synergy Software). Enzyme activity was assessed by measuring the release of reducing sugars over polysaccharide concentration ranges of 0.05-10 mg/mL. Triplicates were collected for each time point at each substrate concentration used throughout the analyses.

Polysaccharide Analysis Using HPLC.

Solutions of xylan or β-glucan (250 μL volume at 1.0 mg/mL) were treated with the Type 1-7.1 or Type 2-8.6 proteins (a 10 μL solution at 22 g protein/mL) for ten and 60 min. Negative controls included xylan and β-glucan substrates without enzyme amendments. Samples were incubated at 45° C., and reactions were terminated by adding 10 μL of 0.5 M NaOH. The 25 μL reactions were then injected onto a DX-500 HPLC instrument (Dionex) equipped with a 250×4 mm CarboPac-1 column (Dionex) at a solvent flow rate of 1 mL min⁻¹. The gradient system utilized 100 mM NaOH as Solvent A and 100 mM NaOH, 1 M NaOAc as Solvent B. The gradient was run at 100% A for 15 min, followed by a linear gradient to 100% B over 60 min. Detection was by pulsed amperometry using an ED40 electrochemical detector (Dionex; 25).

### Example 2

### Results Obtained

Classification of Protozoan Metagenomic cDNAs.

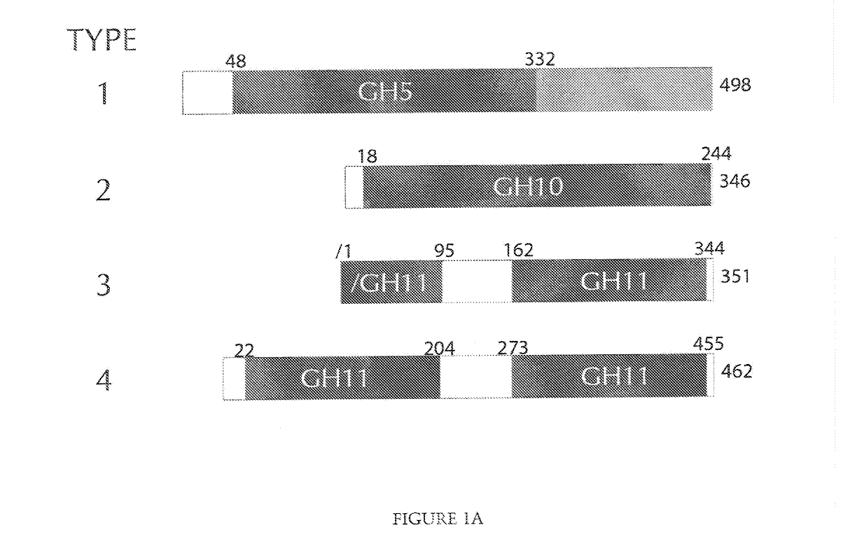

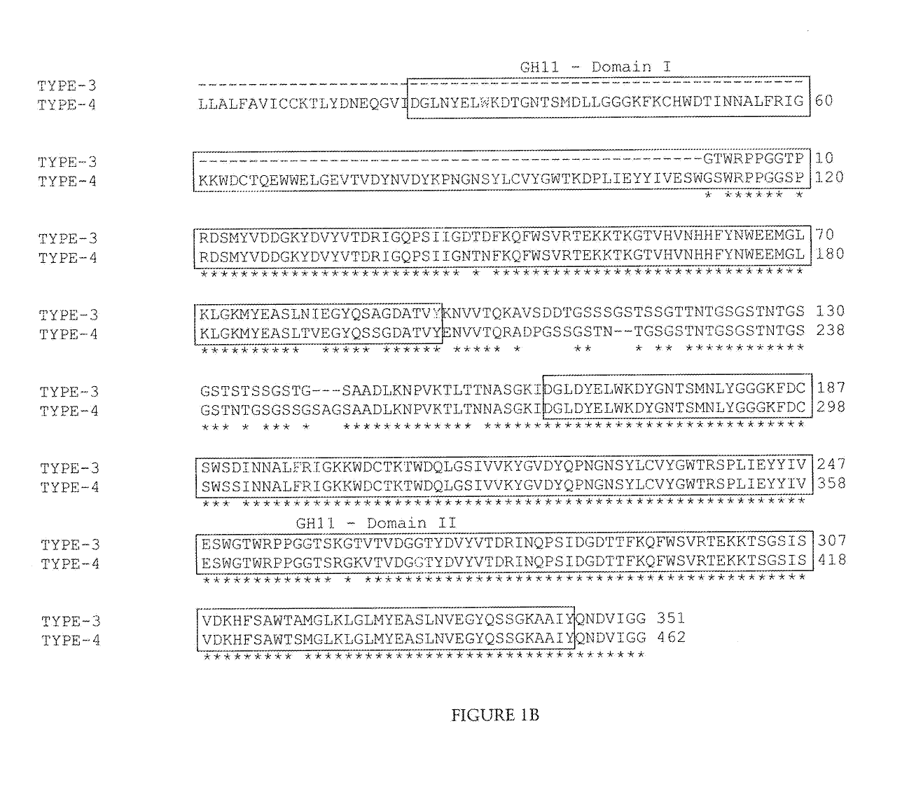

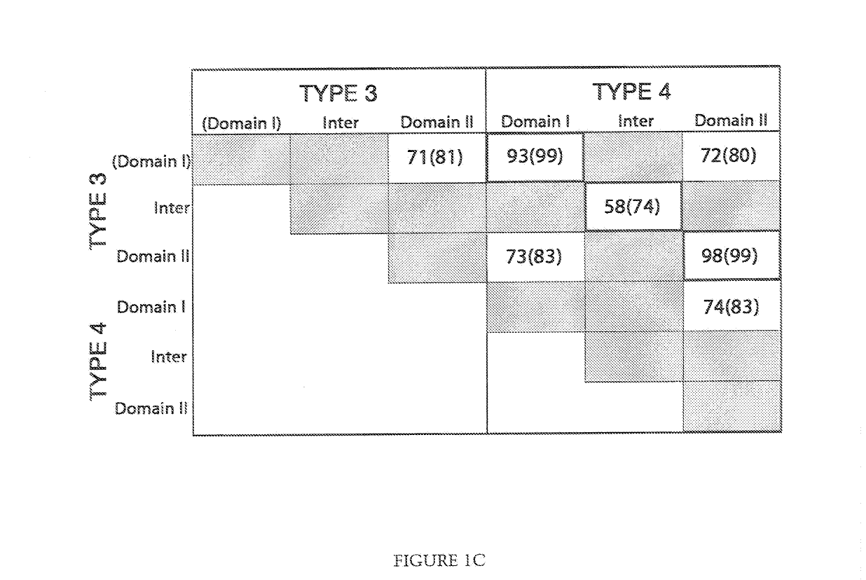

We sequenced a total of 63 clones positive for glycoside hydrolase activity: 60 identified on xylan substrate and three on cellulose substrate. Sequencing of the 5-prime end of each cDNA generated approximately 800 b.p. of sequence for each clone; analysis of these sequences permits classification of the cDNAs into four Types, which are discussed in detail below. Because eight of the 63 cDNAs likely represent aberrant clones (see discussion below and FIG. 5), we have omitted them in our overviews, which are thus restricted to 55 clones (Table 1 and FIG. 1).

TABLE 1
Summary of Metagenomic Screen Positives.

| TYPE | 1 | 2 | 3 | 4 |
|---|---|---|---|---|
| SUBSTRATE | Cellulose | Xylan | Xylan | Xylan |
| CDS | 498 | 346 | 351 | 462 |
| GH DOMAIN(S) | GH5 | GH10 | GH11 + GH11 | GH11 + GH11 |
| PFAM | pfam00150 | pfam00331 | pfam00457 | pfam00457 |
| BEST MATCH | CAH69214 | CAL91981 | CAL91983 | CAL91983 |
| SPECIES | E. ecaudatum | E. ecaudatum | E. ecaudatum | E. ecaudatum |
| % IDENT/% SIM | 83/91 | 82/91 | 67/74 | 70/78 |
| TOTAL cDNAs | 3 | 1 | 2 | 49 |
| UNIQUE cDNAs | 1 | 1 | 1 | 13 |

Four types (“TYPES1-4”, Row 1) of cDNAs were recovered from the activity-based screens, which utilized either cellulose- or xylan-based dye-linked “SUBSTRATE” (Row 2). The length of the coding region (“CDS”, row 3) in amino acids of the longest cDNA for the given “TYPE”. “GH DOMAIN(S)” (row 4), indicates the Glycoside Hydrolase (GH) domain(s) detected by BLASTp homology search; whereas “PFAM” (row 5) indicates the Pfam assignment for the respective GH domain. “BEST MATCH” indicates the GenBank (protein) accession number for the best hits, which were all derived from E. ecaudatum (“SPECIES”, row 7). Percent similarity and identity (“% IDENT % SIM”) to the “BEST MATCH” are indicated (row 8). The total number (row 9) of cDNAs sequenced from each “TYPE” and number of cDNAs with unique 5-prime ends (“UNIQUE cDNAs”, row 10) are indicated.

Cellulase Positives:

The three Type 1 cDNAs were isolated on cellulose indicator plates. Each Type 1 cDNA encodes a single GH5 domain-containing protein (cellulase superfamily; 26); all three cDNA sequences were identical, suggesting that they were independent isolates of the same, amplified cDNA. The Type 1 cDNA sequence is provided as SEQ ID NO:5 and the sequence of the encoded protein is provided as SEQ ID NO:6 in the sequence listing.

Xylanase Positives:

Type 2 through Type 4 cDNAs were isolated on xylan indicator plates. Sequence analysis of these positives with xylanase activity sort into three distinct classes (FIG. 1A): The Type 2 cDNA encodes a protein with a single GH10 domain. The Type 2 cDNA sequence is provided as SEQ ID NO:7 and the sequence of the encoded protein is provided as SEQ ID NO:8 in the sequence listing.

The Type 3 cDNA encodes a partial, N-terminal GH11 domain in addition to a second, full-length GH11 domain; thus, it is unlikely to be a full-length cDNA. The Type 3 cDNA sequence is provided as SEQ ID NO:9 and the sequence of the encoded protein is provided as SEQ ID NO:10 in the sequence listing. The Type 4 cDNAs encode a protein with two complete GH11 domains. The Type 4 cDNA sequence is provided as SEQ ID NO:11 and the sequence of the encoded protein is provided as SEQ ID NO: 12 in the sequence listing. Among the Type 4 clones, DNA sequencing identified 13 different 5-prime ends. The longest cDNA encodes an ORF that contains two GH11 domains; whereas the shortest cDNAs encode at least the C-terminal GH11 domain. In addition to these intact Type 4 cDNAs, we also identified eight cDNAs that likely represent aberrant, deletion forms of the full-length Type 4 cDNA (FIG. 5).

We next compared sequences of the two-domain GH11 positive types (Type 3 and Type 4) through DNA and polypeptide alignments, which indicate that they represent highly similar, yet distinct genes. The DNA alignments (not shown) between the overlapping 1118 b.p. of the two cDNAs show 94.6% identity, with nine gaps, most of which are in the 3-prime untranslated regions of the two cDNAs. A ClustalW protein alignment (FIG. 1B) indicates an overall identity between the two proteins of 92.5%, with two gaps. The best conservation (FIG. 1C) is between the first GH11 domain (“Domain I”) of each protein and between the second GH11 domain (“Domain II”) of each protein. In contrast, comparing the first GH 11 domain of each protein to the GH11 domain of other protein revealed lower conservation, which may indicate a diversification of substrate specificity between the two domains.

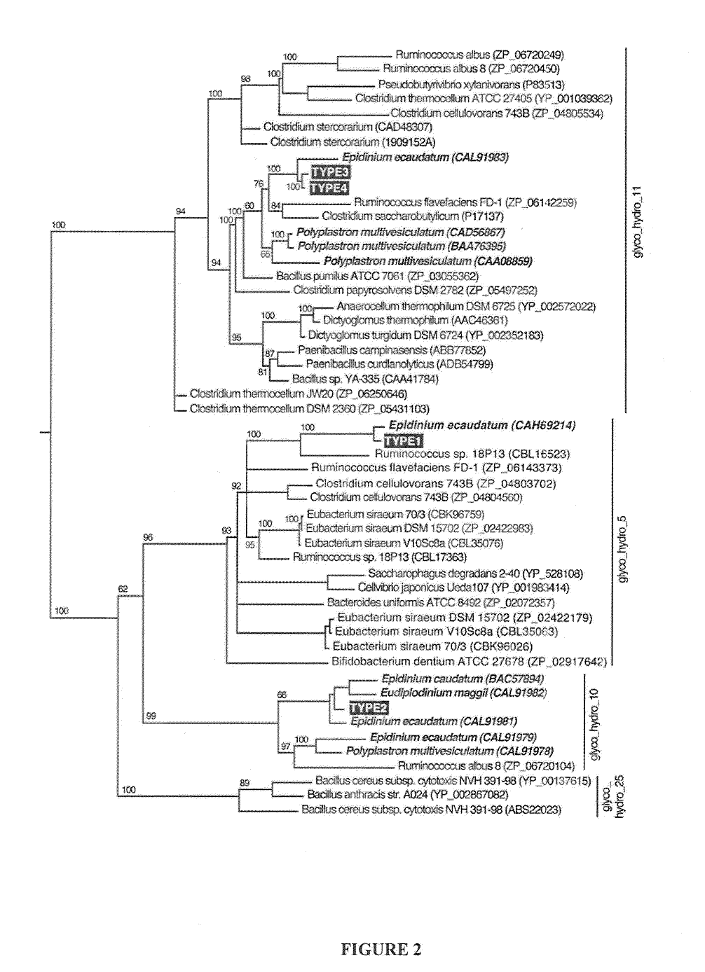

We performed a phylogenetic analysis on the full-length peptide sequences of each positive Type (FIG. 2). The phylogenetic tree was generated by maximum parsimony analysis of the retrieved amino acid sequences and the closest related sequences in the NCBI protein database. The closest related amino acid sequences for all of the searched sequences originated from another protozoan. The 498-residue Type 1-7.1 (cellulose substrate) ORF shares 83% Identity and 91% similarity over 496 residues with a GH5-containing protein sequence (CAH6914) identified from E. ecaudatum. In addition to the GH5-homologous domain (residues 64-335, identified as Pfam00150), this ORF also contains a C-terminal 163 amino acid sequence (residues 336-498) with no significant hit in either BLASTp or Psi-blast searches. Additional analysis would be required to determine the potential role of this novel domain in carbohydrate binding, non-catalytic stabilization, etc. The Type 2-8.6 sequence (putative xylanase) is most closely related to a GH10-domain protein from E. ecaudatum; whereas the dual-domain Type 3 and 4 proteins are most closely related to a different dual GH11 domain sequence, also identified in E. ecaudatum. Thus, the phylogenetic analysis suggests that the amino acid sequences identified in this study were of protozoan, not bacterial, origin. In further support of this, cDNAs for the shorter Types (Types 1-3), as well as the full-length sequence of the Type 4, have polyA tracts; Types 1-3 also had a typical eukaryotic upstream polyadenylation signal (AATAAA). As has been reported for numerous other ruminal protozoan genes (14, 15, 17, 37), the codon usage analysis (data not shown) indicates a strong bias for A and T nucleotides in the first and third positions, as is also reflected by the G+C content (32-36%) of the nucleotide sequences of all four types analyzed. Notably, the closest non-protozoan homolog for each Type included Ruminococcus species, a group that includes anaerobic, cellulolytic bacteria.

### Example 3

### Biochemical Characterization of the Type 1-7.1 Enzyme

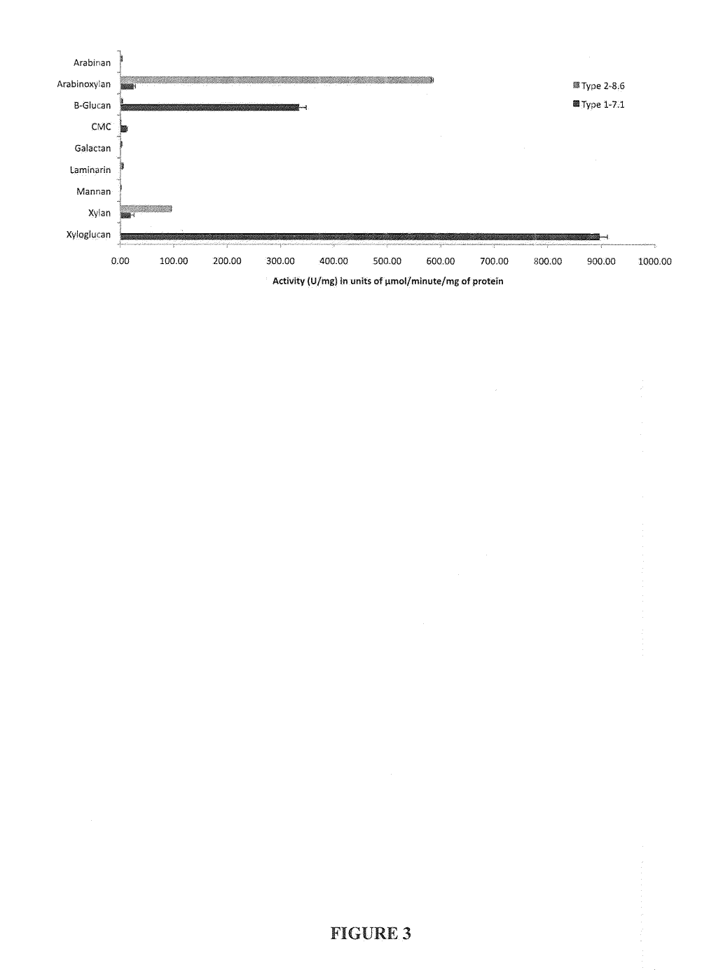

The Type 1-7.1 positive enzyme was identified as a possible cellulase due to its activity on cellulose indicator plates and its GH5 domain homology. While many characterized cellulases are typically active against carboxymethyl cellulose (CMC), the recombinant enzyme derived from our library and comprising the protein of SEQ ID NO:6 had 85 times higher activity against xyloglucan (896.06±14.98 U/mg) compared against CMC (10.45±2.03 U/mg) and 32 times the activity against β-glucan (334.29±13.92 U/mg) (FIG. 3). It also exhibited minimal activity against arabinoxylan (24.39±3.64 U/mg) and xylan (19.09±6.49 U/mg). Furthermore, no activity was detected against arabinan, galactan, laminarin, or mannan (FIG. 3). When the Type 1-7.1 amino acid sequence was compared to its closest BLASTp match (CAH69214) (48), a cellulase identified in E. ecaudatum, it exhibited additional differences. The pH optimum for the cellulase from E. ecaudatum was reported to be 8.3 (53); whereas the optimum for Type 1-7.1 is 5.9 (Table 2 and FIGS. 7A and 7B). The apparent Kₘ, Vₘₐₓ, and K_{cat}, and K_{cat}/Kₘ for β-glucan are 0.83 mg/ml, 97.7 μmol/min/mg, 7.4 s⁻¹, and 8.9 ml mg⁻¹ s⁻¹, and for xyloglucan are 0.19 mg/ml, 179.1 mol/min/mg, 13.6 s⁻¹, and 71.6 ml mg⁻¹ s⁻¹, respectively (Table 3). The Kₘ value for xyloglucan was slightly lower than the Kₘ value for β-glucan, indicating a slightly higher affinity for this substrate. Moreover, the Vₘₐₓ value for xyloglucan was 2.3 times higher than for β-glucan. The enzyme turnover rate, K_{cat}, and the catalytic efficiency (k_{cat}/Kₘ) were also higher for xyloglucan. It is possible that this enzyme might be considered a specific xyloglucanase because its activity against xyloglucan is more than ten-times higher than its activity against CMC (24). However, further analyses are required for confirmation. Xyloglucanases have been also identified in fungi and bacteria (22, 23, 24, 40, 56). Due to its broad pH and temperature tolerances (Table 2, FIGS. 7A and C), our xyloglucanase could be useful in industrial degradation of hemicelluloses from plant biomass.

TABLE 2
Basic Biochemical Properties of Type 1-
7.1 and Type 2-8.6 Recombinant Proteins.

| Property | Type 1-7.1 | Type 2-8.6 |
|---|---|---|
| GH Family | 5 | 10 |
| Molecular Mass (kDa) | 56.0 | 39.2 |
| pI (calculated) | 4.56 | 6.35 |
| pH activity range | 5.0 to 8.0 | 5.0 to 9.0 |
| pH optimum | 5.9 | 7.4 |
| Temperature activity range | 4° C. to 60° C. | 25° C. to 60° C. |
| Temperature optimum | 50° C. | 45° C. |

TABLE 3
Kinetic Data Type 1-7.1 and Type 2-8.6 Recombinant Proteins¹.

| | | | | Catalytic |
|---|---|---|---|---|
| | Apparent | | | Efficiency |
| | Kₘ | Vₘₐₓ | K_{cat} | (K_{cat}/Kₘ) |
| Type 1-7.1 | | | | |
| Protein Substrate | | | | |
| β-glucan | 0.83 mg/ml | 97.7 μmol/min/mg | 7.4 (s⁻¹) | 8.9 ml mg⁻¹ s⁻¹ |
| Xyloglucan | 0.19 mg/ml | 179.1 μmol/min/mg | 13.6 (s⁻¹) | 71.6 ml mg⁻¹ s⁻¹ |
| Type 2-8.6 | | | | |
| Protein Substrate | | | | |
| Xylan | 6.95 mg/ml | 14.5 μmol/min/mg | 1.1 (s⁻¹) | 0.2 ml mg⁻¹ s⁻¹ |
| Arabinoxylan | 0.14 mg/ml | 117.3 μmol/min/mg | 8.9 (s⁻¹) | 63.6 ml mg⁻¹ s⁻¹ |

¹The Type 1-7.1 protein comprised the protein sequence of SEQ ID NO: 6 and the Type 2-8.6 protein comprised the sequence of SEQ ID NO: 8.

### Example 4

### Biochemical Characterization of the Type 2-8.6 Enzyme

The closest BLASTp match for Type 2-8.6 enzyme was also a GH10 domain-containing enzyme. Typically, members of this GH tend to have low pI values (9). In contrast, Type 2-8.6 has a calculated pI value of 6.35 (Table 2), a value that was higher than those reported for GH 10 enzymes, but a lower value than what is typically observed for GH 11 enzymes (which tend to be high values; reference 9). Although the enzyme had detectable activity against xylan (95.62±0.38 U/mg), it possessed a higher activity against arabinoxylan (584.39±2.07 U/mg) (FIG. 3). The apparent Kₘ, Vₘₐₓ, K_{cat}, and K_{cat}/Kₘ, for xylan are 6.95 mg/ml, 14.5 μmol/min/mg, 1.1 s⁻¹, and 0.2 ml mg⁻¹ s⁻¹, and for arabinoxylan are 0.14 mg/ml, 117.3 mol/min/mg, 8.9 s⁻¹, and 63.6 ml mg⁻¹ s⁻¹, respectively (Table 3). While enzymes that possess activity against arabinoxylan have been characterized in bacteria, fungi, and plants (11), no protozoan enzymes have been identified to date. To confirm the complete enzymatic hydrolysis of arabinoxylan, further studies need to be completed to determine the reaction products and structure of the enzyme (e.g., 10).

### Example 5

### Mode of Action of the Type 1-7.1 and Type 2-8.6 Enzymes

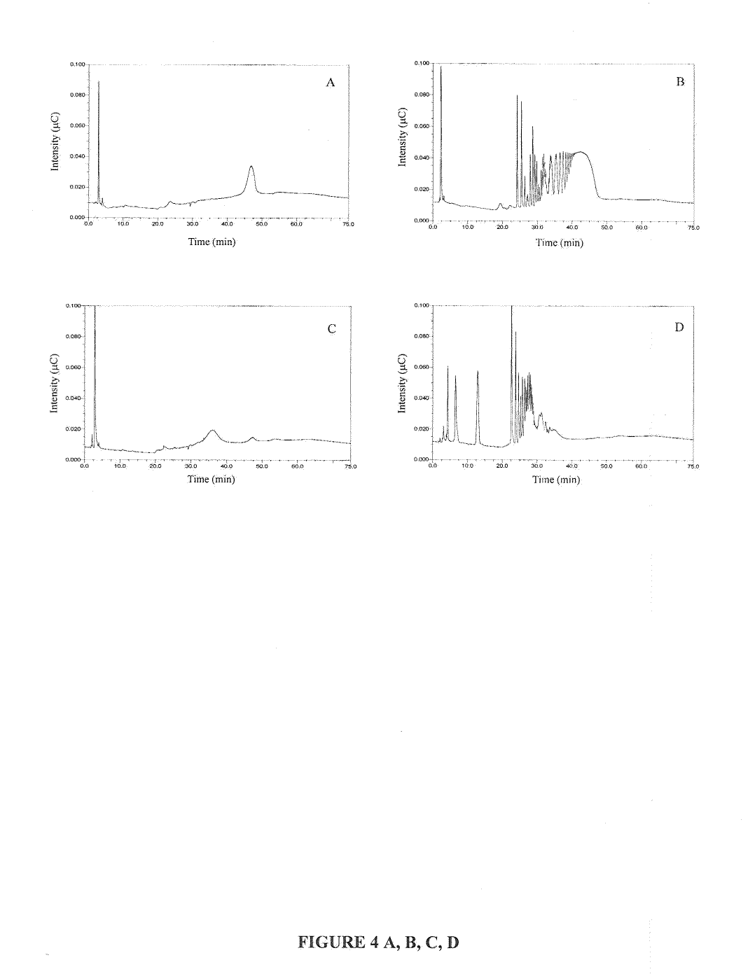

Fibrolytic enzymes are essential for the digestion of cellulosic biomass in the ruminant diet. A suite of enzymes is required to produce a variety of free sugars, (exo-enzymes), as well as oligosaccharides (endo-enzymes) for metabolism by rumen bacteria and subsequent digestion by the ruminant host. The HPLC analysis of hydrolysis products resulting from exposure of β-glucan to Type 1-7.1 enzyme and xylan exposure to Type 2-8.6 indicated that each tested enzyme employs an endo-type of cleavage (FIG. 4). When compared to chromatographs of β-glucan (FIG. 4A) and xylan (FIG. 4C), not exposed to the recombinant enzymes, each of the treated substrates had multiple peaks prior to the polysaccharides peaks (FIGS. 4 B and D), indicating that a range of oligosaccharides was generated by the actions of the endo-glycoside hydrolases. It should be noted that similar chromatographs were obtained for both 10 and 60 minute exposures. If the enzymes were exo-acting, the primary products would be either glucose (from β-glucan) or xylose (from xylan). Both glucose and xylose formed a single peak at 4 min on the chromatographs (data not shown). There was no peak at the 4 min point for glucose when β-glucan was exposed to Type 1-7.1, yet multiple peaks were present as the chromatographic run proceeded (FIG. 4B). There was a peak at 4 min, indicating xylose production after xylan exposure to Type 2-8.6, as well as multiple peaks at later time points, indicating the presence of oligoxylose chains (FIG. 4D). These data are consistent with the fact that many GH5 and GH10 enzymes are known to be endo-β-1,4-glucanases and endo-β-1,4-xylanases (9).

### Example 6

### Discussion of Results

We executed an activity-based metagenomic screen with the aim of assessing the diversity of fibrolytic enzymes encoded by the meta-transcriptome of protozoa present in bovine rumen fluid. Using just two substrates, a cellulose and a hemicellulose, we identified four genes with diverse GH domains and modular organization. Phylogenetic analysis of these genes revealed the closest homologs to be protozoan, and the closest non-protozoan homologs to be most closely related to gram-positive bacteria. These observations support the hypothesis that lignocellulose-degrading genes were acquired by protozoa from ruminal bacteria by horizontal gene transfer (15, 41). There was close homology of the positive sequences obtained in our study (FIG. 1B) to E. ecaudatum genes identified in a previous protozoan EST sequencing study (41). The putative cellulases and xylanases identified by Ricard et al. (41) were all derived from one (the Entodiniophorphids) of the two major groups of rumen ciliates. The fact that each of the gene types identified in our bovine rumen screens had potential homologs in protozoan species originating from sheep rumen raises the possibly that the genes represent orthologs derived from related ciliate species in the two different hosts. The fact that none of our positives exhibited novel domain organization (relative to other identified ciliate GH genes) may indicate that ruminal ciliates may have acquired only a limited repertoire of bacterial fibrolytic genes.

One strength of activity-based screening is its ability to directly recover genes encoding biocatalysts for specific substrates (e.g., cellulose). In our current study, we identified, expressed and biochemically characterized two enzymes. The putative xyloglucanase possesses a high specific activity towards tamarind xyloglucan (896.06 U/mg protein) (FIG. 3). In comparison, previously characterized xyloglucanases from fungal sources ranged in their activity from 45 to 98 U/mg protein (24). Our xyloglucanase also had a lower apparent Kₘ value (0.19 mg/ml) (Table 3) than a xyloglucan-specific endo-β-1,4-glucanase gene (xeg5A), isolated from a cow's rumen microflora and expressed in E. coli, (3.61 mg/ml) (56) and one isolated from the fungus, Aspergillus aculeatus, (3.6 mg/ml) (40). Similarly, our putative arabinoxylanase had a lower apparent Kₘ (0.14 mg arabinoxylan/ml) (Table 3) than a GH10 xylanase isolated from P. funiculoszum (3.7 mg/ml) (34). In addition, our putative arabinoxylanase had a higher specific activity (584.39 U/mg) (FIG. 3) than the P funiculosum xylanase (106.4 U/mg) (34) on arabinoxylan, as well as a higher specific activity against Beechwood xylan (Type 2-8.6, 95.62 U/mg; P. funiculosum xylanase, 60.1 U/mg) (34).

The powerful and rapid, activity-based metagenomics approach does have its own technical and efficiency obstacles (as discussed by 52), however, as evidenced by our recovery of hybrid/deletion cDNA clones. Thus, more direct, sequence-based metagenomics (e.g., single-cell-based genomic sequencing; reference 57), in combination with molecular phylogenetics (e.g., 13, 48) may represent the more attractive technique for characterizing the population dynamics, functions and fibrolytic genes of ciliate ruminal protozoa.

Having illustrated and described the principles of the present invention, it should be apparent to persons skilled in the art that the invention can be modified in arrangement and detail without departing from such principles. As various modifications could be made in the compositions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

It should also be understood that when introducing elements of the present invention in the claims or in the above description of exemplary embodiments of the invention, the terms “comprising,” “including,” “containing”, and “having” are intended to be open-ended and mean that there may be additional elements other than the listed elements.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

### REFERENCES

1. Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res 25:3389-3402.
2. Anthon, G. E., and D. M. Barrett. 2002. Determination of reducing sugars with 3-methyl-2-benzothiazolinonehydrazone. Anal Biochem 305:287-289.
3. Bailey, R. W., R. T. Clarke, and D. E. Wright. 1962. Carbohydrases of the rumen ciliate Epidinium ecaudatum (Crawley). The Biochemical journal 83:517-523.
4. Bera-Maillet, C., E. Devillard, M. Cezette, J. P. Jouany, and E. Forano. 2005. Xylanases and carboxymethylcellulases of the rumen protozoa Polyplastron multivesiculatum, Eudiplodinium maggii and Entodinium sp. FEMS Microbiol Lett 244:149-156.
5. Britton, H. T. S., and R. A. Robinson. 1931. Universal buffer solutions and the dissociation constant of veronal. Journal of the Chemical Society (Resumed):1456-1462.
6. Chema, R., H. Sugawara, T. Koike, R. Lopez, T. J. Gibson, D. G. Higgins, and J. D. Thompson. 2003. Multiple sequence alignment with the Clustal series of programs. Nucleic Acids Res 31:3497-3500.
7. Clamp, M., J. Cuff, S. M. Searle, and G. J. Barton. 2004. The Jalview Java alignment editor. Bioinformatics 20:426-427.
8. Clayet, F., J. Senaud, and J. Bohatier. 1992. Chromatographic separation of some cell wall polysaccharide-degrading enzymes of the sheep rumen Ciliate Epidinium caudatum. Ann Zootech 41:81.
9. Collins, T., C. Gerday, and G. Feller. 2005. Xylanases, xylanase families and extremophilic xylanases. FEMS Microbiol Rev 29:3-23.
10. Correia, M. A., K. Mazumder, J. L. Bras, S. J. Firbank, Y. Zhu, R. J. Lewis, W. S. York, C. M. Fontes, and H. J. Gilbert. 2011. Structure and function of an arabinoxylan-specific xylanase. The Journal of biological chemistry 286:22510-22520.
11. Courtin, C. M., and J. A. Delcour. 2002. Arabinoxylans and endoxylanases in wheat flour bread-making. Journal of Cereal Science 35:225-243.
12. Dehority, B. A. 1993. Laboratory manual for classification and morphology of ruminal ciliate protozoa. CRC Press, Boca Raton, Fla.
13. Deng, W., D. Xi, H. Mao, and M. Wanapat. 2008. The use of molecular techniques based on ribosomal RNA and DNA for rumen microbial ecosystem studies: a review. Mol Biol Rep 35:265-274.
14. Devillard, E., C. Bera-Maillet, H. J. Flint, K. P. Scott, C. J. Newbold, R. J. Wallace, J. P. Jouany, and E. Forano. 2003. Characterization of XYN00B, a modular xylanase from the ruminal protozoan Polyplastron multivesiculatum, with a family 22 carbohydrate-binding module that binds to cellulose. Biochem J 373:495-503.
15. Devillard, E., C. J. Newbold, K. P. Scott, E. Forano, R. J. Wallace, J. P. Jouany, and H. J. Flint. 1999. A xylanase produced by the rumen anaerobic protozoan Polyplastron multivesiculatum shows close sequence similarity to family 11 xylanases from gram-positive bacteria. FEMS Microbiol Lett 181:145-152.
16. Edgar, R. C. 2004. MUSCLE: multiple sequence alignment with high accuracy and high throughput. Nucleic Acids Res 32:1792-1797.
17. Eschenlauer, S. C., N. R. McEwan, R. E. Calza, R. J. Wallace, R. Onodera, and C. J. Newbold. 1998. Phylogenetic position and codon usage of two centrin genes from the rumen ciliate protozoan, Entodinium caudatum. FEMS Microbiol Lett 166:147-154.
18. Felsenstein, J. 2000. PHYLIP (Phylogeny Inference Package). Department of Genetics, University of Washington., Seattle.
19. Fernandez-Arrojo, L., M. E. Guazzaroni, N. Lopez-Cortes, A. Beloqui, and M. Ferrer. 2010. Metagenomic era for biocatalyst identification. Curr Opin Biotechnol 21:725-733.
20. Ferrer, M., O. V. Golyshina, T. N. Chemikova, A. N. Khachane, D. Reyes-Duarte, V. A. Santos, C. Strompl, K. Elborough, G. Jarvis, A. Neef, M. M. Yakimov, K. N. Timmis, and P. N. Golyshin. 2005. Novel hydrolase diversity retrieved from a metagenome library of bovine rumen microflora. Environ Microbiol 7:1996-2010.
21. Gijzen, H. J., H. J. Lubberding, M. J. T. Gerhardus, and G. D. Vogels. 1988. Contribution of rumen protozoa to fibre degradation and cellulase activity in vitro. FEMS Microbiology Letters 53:35-43.
22. Gilbert, H. J., H. Stalbrand, and H. Brumer. 2008. How the walls come crumbling down: recent structural biochemistry of plant polysaccharide degradation. Curr Opin Plant Biol 11:338-348.
23. Gloster, T. M., F. M. Ibatullin, K. Macauley, J. M. Eklof, S. Roberts, J. P. Turkenburg, M. E. Bjornvad, P. L. Jorgensen, S. Danielsen, K. S. Johansen, T. V. Borchert, K. S. Wilson, H. Brumer, and G. J. Davies. 2007. Characterization and three-dimensional structures of two distinct bacterial xyloglucanases from families GH5 and GH12. J Biol Chem 282:19177-19189.
24. Grishutin, S. G., A. V. Gusakov, A. V. Markov, B. B. Ustinov, M. V. Semenova, and A. P. Sinitsyn. 2004. Specific xyloglucanases as a new class of polysaccharide-degrading enzymes. Biochim Biophys Acta 1674:268-281.
25. Hausalo, T. 1995. Analysis of wood and pulp carbohydrates by anion exchange chromatography with pulse amperometric detection., 8th International Symposium on Wood and Pulping Chemistry, Helsinki, Finland.
26. Henrissat, B. 1991. A classification of glycosyl hydrolases based on amino acid sequence similarities. Biochem J 280 (Pt 2):309-316.
27. Henrissat, B., and G. J. Davies. 2000. Glycoside hydrolases and glycosyltransferases. Families, modules, and implications for genomics. Plant Physiol 124:1515-1519.
28. Hess, M., A. Sczyrba, R. Egan, T. W. Kim, H. Chokhawala, G. Schroth, S. Luo, D. S. Clark, F. Chen, T. Zhang, R. I. Mackie, L. A. Pennacchio, S. G. Tringe, A. Visel, T. Woyke, Z. Wang, and E. M. Rubin. 2011. Metagenomic discovery of biomass-degrading genes and genomes from cow rumen. Science 331:463-467.
29. Howard, B. H., G. Jones, and M. R. Purdom. 1960. The pentosanases of some rumen bacteria. The Biochemical journal 74:173-180.
30. Huang, X., and A. Madan. 1999. CAP3: A DNA sequence assembly program. Genome Res 9:868-877.
31. Karlin, S., and S. F. Altschul. 1990. Methods for assessing the statistical significance of molecular sequence features by using general scoring schemes. Proc Natl Acad Sci USA 87:2264-2268.
32. Kim, M., M. Morrison, and Z. Yu. 2011. Status of the phylogenetic diversity census of ruminal microbiomes. FEMS Microbiol Ecol 76:49-63.
33. Krause, D. O., S. E. Denman, R. I. Mackie, M. Morrison, A. L. Rae, G. T. Attwood, and C. S. McSweeney. 2003. Opportunities to improve fiber degradation in the rumen: microbiology, ecology, and genomics. FEMS Microbiol Rev 27:663-693.
34. Lafond, M., A. Tauzin, V. Desseaux, E. Bonnin, H. Ajandouz el, and T. Giardina. 2011. GH10 xylanase D from Penicillium funiculosum: biochemical studies and xylooligosaccharide production. Microb Cell Fact 10:20.
35. Li, L. L., S. R. McCorkle, S. Monchy, S. Taghavi, and D. van der Lelie. 2009. Bioprospecting metagenomes: glycosyl hydrolases for converting biomass. Biotechnol Biofuels 2:10.
36. Martin, C., A. G. Williams, and B. Michalet-Doreau. 1994. Isolation and characteristics of the protozoal and bacterial fractions from bovine ruminal contents. J Anim Sci 72:2962-2968.
37. McEwan, N. R., S. C. Eschenlauer, R. E. Calza, R. J. Wallace, and C. J. Newbold. 2000. The 3′ untranslated region of messages in the rumen protozoan Entodinium caudatum. Protist 151:139-146.
38. Michalowski, T., K. Rybicka, K. Wereszka, and A. Kasperowicz. 2001. Ability of the rumen ciliate Epidinium ecaudatum to digest and use crystalline cellulose and xylan for in vitro growth. Acta Protozoologica 40:203-210.
39. Palackal, N., C. S. Lyon, S. Zaidi, P. Luginbuhl, P. Dupree, F. Goubet, J. L. Macomber, J. M. Short, G. P. Hazlewood, D. E. Robertson, and B. A. Steer. 2007. A multifunctional hybrid glycosyl hydrolase discovered in an uncultured microbial consortium from ruminant gut. Appl Microbiol Biotechnol 74:113-124.
40. Pauly, M., L. N. Andersen, S. Kauppinen, L. V. Kofod, W. S. York, P. Albersheim, and A. Darvill. 1999. A xyloglucan-specific endo-beta-1,4-glucanase from Aspergillus aculeatus: expression cloning in yeast, purification and characterization of the recombinant enzyme. Glycobiology 9:93-100.
41. Ricard, G., N. R. McEwan, B. E. Dutilh, J. P. Jouany, D. Macheboeuf, M. Mitsumori, F. M. McIntosh, T. Michalowski, T. Nagamine, N. Nelson, C. J. Newbold, E. Nsabimana, A. Takenaka, N. A. Thomas, K. Ushida, J. H. Hackstein, and M. A. Huynen. 2006. Horizontal gene transfer from Bacteria to rumen Ciliates indicates adaptation to their anaerobic, carbohydrates-rich environment. BMC Genomics 7:22.
42. Riesenfeld, C. S., P. D. Schloss, and J. Handelsman. 2004. Metagenomics: genomic analysis of microbial communities. Annu Rev Genet. 38:525-552.
43. Rubin, E. M. 2008. Genomics of cellulosic biofuels. Nature 454:841-845.
44. Ruijssenaars, H. J., and S. Hartmans. 2001. Plate screening methods for the detection of polysaccharase-producing microorganisms. Appl Microbiol Biotechnol 55:143-149.
45. Russell, J. B., and J. L. Rychlik. 2001. Factors that alter rumen microbial ecology. Science 292:1119-1122.
46. Schmidt, H. A., K. Strimmer, M. Vingron, and A. von Haeseler. 2002. TREE-PUZZLE: maximum likelihood phylogenetic analysis using quartets and parallel computing. Bioinformatics 18:502-504.
47. Shin, E. C., K. M. Cho, W. J. Lim, S. Y. Hong, C. L. An, E. J. Kim, Y. K. Kim, B. R. Choi, J. M. An, J. M. Kang, H. Kim, and H. D. Yun. 2004. Phylogenetic analysis of protozoa in the rumen contents of cow based on the 18S rDNA sequences. J Appl Microbiol 97:378-383.
48. Sylvester, J. T., S. K. Karnati, Z. Yu, M. Morrison, and J. L. Firkins. 2004. Development of an assay to quantify rumen ciliate protozoal biomass in cows using real-time PCR. J Nutr 134:3378-3384.
49. Takenaka, A., C. G. D'Silva, H. Kudo, H. Itabashi, and K. J. Cheng. 1999. Molecular cloning, expression, and characterization of an endo-beta-1,4-glucanase cDNA from Epidinium caudatum 1. J Gen Appl Microbiol 45:57-61.
50. Takenaka, A., K. Tajima, M. Mitsumori, and H. Kajikawa. 2004. Fiber digestion by rumen ciliate protozoa. Microbes and Environments 19:203-210.
51. Ten, L. N., W. T. Im, Z. Aslam, L. Larina, and S. T. Lee. 2007. Novel insoluble dye-labeled substrates for screening inulin-degrading microorganisms. J Microbiol Methods 69:353-357.
52. Uchiyama, T., and K. Miyazaki. 2009. Functional metagenomics for enzyme discovery: challenges to efficient screening. Curr Opin Biotechnol 20:616-622.
53. Wereszka, K., F. M. McIntosh, T. Michalowski, J. P. Jouany, E. Nsabimana, D. Macheboeuf, N. R. McEwan, and C. J. Newbold. 2004. A cellulase produced by the rumen anaerobic protozoan epidinium ecaudatum has an unusual pH optimum. Endocytobiosis and Cell Research 15:561-569.
54. Williams, A. G., and G. S. Coleman. 1985. Hemicellulose-degrading enzymes in rumen ciliate protozoa. Current Microbiology 12:85-90.
55. Williams, A. G., and G. S. Coleman. 1992. The Rumen Protozoa. Springer-Verlag, New York, N.Y.
56. Wong, D. D., V. J. Chan, A. A. McCormack, and S. B. Batt. 2010. A novel xyloglucan-specific endo-beta-1,4-glucanase: biochemical properties and inhibition studies. Appl Microbiol Biotechnol 86:1463-1471.
57. Woyke, T., G. Xie, A. Copeland, J. M. Gonzalez, C. Han, H. Kiss, J. H. Saw, P. Senin, C. Yang, S. Chatterji, J. F. Cheng, J. A. Eisen, M. E. Sieracki, and R. Stepanauskas. 2009. Assembling the marine metagenome, one cell at a time. PLoS One 4:e5299.
58. Zmasek, C. M., and S. R. Eddy. 2001. ATV: display and manipulation of annotated phylogenetic trees. Bioinformatics 17:383-384.

What is claimed is:

1. A recombinant nucleic acid comprising a heterologous promoter that is operably linked to a gene encoding a protein having at least 85% identity to SEQ ID NO: 6 and glycosidase activity.

2. The recombinant nucleic acid of claim 1, wherein nucleic acid is DNA.

3. The recombinant nucleic acid of claim 2, said promoter provides for expression of the glycosidase in a bacterial cell, a yeast cell, a plant cell, a fungal cell, an algal cell, a protozoan cell, or a mammalian cell.

4. The recombinant nucleic acid of claim 1, wherein said glycosidase comprises at least one GH5 domain.

5. The recombinant nucleic acid of claim 1, wherein said glycosidase activity comprises a xyloglucanase activity.

6. The recombinant nucleic acid of claim 1, wherein said protein has at least 90%, 95%, 98%, 99%, or 100% sequence identity to SEQ ID NO:6.

7. A transformed cell comprising the recombinant nucleic acid of claim 1.

8. The transformed cell of claim 7, wherein said cell is a bacterial cell, a yeast cell, an algal cell, a protozoan cell, a plant cell, a fungal cell, or a mammalian cell.

9. The transformed cell of claim 7, wherein said promoter provides for constitutive and/or inducible expression of the protein in the cell.

10. A method of making a glycosidase comprising the steps of:
c. culturing the transformed cell of claim 7 under conditions that provide for accumulation of the protein in the cell or in the cell culture medium; and,
d. harvesting said protein from said cell or said cell culture medium.

11. A method of degrading hemicellulose comprising culturing the transformed cell of claim 7 in the presence of hemicellulose under conditions that provide for accumulation of the protein in the cell or in the cell culture medium and for at least partial hydrolysis of xyloglucans in said hemicellulose.

12. The method of claim 11, wherein said hemicellulose is obtained from plant biomass.

13. The method of claim 12, wherein said plant biomass is selected from the group consisting of corn fiber, corn stover, wheat straw, rice straw, rice bran, switchgrass, wood, and sugarcane bagasse.

14. An isolated protein having at least 85% identity to SEQ ID NO: 6 and glycosidase activity.

15. A method for degrading hemicellulose comprising incubating a protein having at least 85% identity to SEQ ID NO: 6 and glycosidase activity with hemicellulose in a reaction vessel under conditions that provide for at least partial hydrolysis of xyloglucans in said hemicellulose.

16. The method of claim 15, wherein said conditions comprise a temperature range of about 4° C. to about 60° C.

17. The method of claim 16, wherein said conditions comprise a temperature range of about 35° C. to about 60° C.

18. The method of claim 15, wherein said conditions comprise a pH of about 5.0 to about 8.0.

19. The method of claim 15, wherein said hemicellulose is obtained from plant biomass.

20. The method of claim 19, wherein said plant biomass is selected from the group consisting of corn fiber, corn stover, wheat straw, rice straw, switchgrass, wood, and sugarcane bagasse.
